(19) United States
(12) Patent Application Publication
(10) Pub. No.: US 2023/0331679 A1
(43) Pub. Date: Oct. 19, 2023

(54) NAPHTHALENE MONOIMIDE COMPOUNDS AND METHODS THEREOF

(71) Applicant: JAWAHARLAL NEHRU CENTRE FOR ADVANCED SCIENTIFIC RESEARCH, Bangalore (IN)

(72) Inventors: Govindaraju THIMMAIAH, Bangalore (IN); Rajasekhar KOLLA, Bangalore (IN); Sourav SAMANTA, Bangalore (IN)

(73) Assignee: JAWAHARLAL NEHRU CENTRE FOR ADVANCED SCIENTIFIC RESEARCH, Bangalore (IN)

(21) Appl. No.: 18/006,578

(22) PCT Filed: Jul. 22, 2021
(86) PCT No.: PCT/IB2021/056633

§ 371 Date: Jan. 24, 2023

(30) Foreign Application Priority Data
Jul. 24, 2020 (IN) 202041031875

(51) Int. Cl. C07D 217/24; A61K 45/06; A61P 25/28; A61K 47/64

(52) U.S. Cl. CPC C07D 217/24; A61K 45/06; A61K 47/64; A61P 25/28

(57) ABSTRACT

The present disclosure discloses a compound of Formula (I) and its polymorphs, stereoisomers, prodrugs, solvates, co-crystals, intermediates, pharmaceutically acceptable salts, and metabolites thereof and a process of preparation of compounds of Formula (I). The present disclosure also discloses a method of treatment of a condition mediated by aggregation of Aβ42, tau, or α-syn. The present disclosure also discloses a compound of Formula (I) that provides reversal or improvement of cognitive decline.

### FIELD OF INVENTION

The subject matter disclosed herein relates to compounds of Formula (I). The subject matter in particular relates to naphthalene monoimide (NMI) compounds of Formula (I). The subject matter further relates a field of drugs for disorders of the nervous system and in particular relates to a pharmaceutically active compound for use in the treatment of neurodegenerative diseases.

### BACKGROUND OF THE INVENTION

Neurodegenerative disease is an umbrella term for a range of incurable diseases caused due to progressive degeneration of neurons/nerve cells which act as building blocks of the central nervous system. Any damage or death of neurons leads to the development of brain related disorders which severely affects an individual’s speech, memory (dementia), and movement (ataxias) capabilities.

Alzheimer’s disease is the most common neurogenerative diseases, while other include tauopathies, Parkinson’s disease Huntington’s disease (HD), Prion disease and Amyotrophic Lateral Sclerosis (ALS), etc. These diseases are highly debilitating which worsen rapidly with increasing age. There are currently ~50 million people suffering from AD, and this number is expected to cross over 130 million by 2050. Besides, the statistics reveal that the number of deaths by AD increased by ~146% between 2000 and 2018, while all other major diseases such as heart diseases, HIV, and cancer showed an appreciable decrease owing to the availability of improved detection and treatment option.

Neurodegeneration has been shown to have a common mechanism of disease pathogenesis occuring majorly due to protein misfolding and aggregation. Under disease conditions, protein and their processed products (peptides) folds abnormally and self-aggregates to form aggregation species through β-sheet (hydrogen bonding) formation, further supported by electrostatic, hydrophobic and van der Waals interactions. These aggregation species accumulate in the brain as Aβ plaques and neuronal inclusions which results in multifaceted toxicity. The aggregation species damage neuronal cell membrane causing cell death, which disrupts the neuron signaling pathways. The protein aggregation is at the core of the disease progression and pathogenesis, and therefore targeting protein aggregation is considered a promising strategy to develop therapeutic agents for these neurodegenerative disorders.

Till date, there have been constant efforts towards developing neuroprotective compounds which can help to retain neuronal health and prevent neuronal loss. AU2011305315A1 discloses a group of unsaturated carbonyl compounds as inhibitors of de-ubiquitinase which aid in treating neurodegenerative diseases. AU2014274253A1 discloses a polypeptide comprising variants of amino acids having an ability to bind and/or disaggregate amyloid resulting in the prevention of diseases associated with misfolding or aggregation of amyloid.

Despite the extensive research done in this field, the presently available therapeutics include vaccines and protein therapies which are associated with various concerns related to their manufacturing cost, drug administration and low patient compliance. Thus, there is still a dire need in the state of art to develop neuroprotective compounds based on a better understanding of neurodegeneration mechanism and protein molecular interactions, which show long term therapeutic effects, does not exhibit harmful side-effects, and at the same time, can be easily obtained and are cost-effective.

### SUMMARY OF THE INVENTION

In a first aspect of the present disclosure, there is provided a compound of Formula (I)

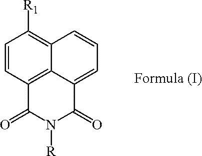

and its polymorphs, stereoisomers, prodrugs, solvates, co-crystals, intermediates, pharmaceutically acceptable salts, and metabolites thereof, wherein
R is selected from C₁₋₁₈ alkyl, C₂₋₁₈ alkenyl, C₂₋₁₈ alkynyl, C₅₋₁₀ aryl, C₁₋₁₈ heteroalkyl, C₅₋₁₈ heteroaryl, C₃₋₁₂ cycloalkyl, C₃₋₁₂ heterocyclyl, -NR₃R₄, -N⁺R₃R₄R₅, -OC₁₋₁₈ alkyl, amino acids, peptides, nucleobases, sugars, or lipids, wherein C₁₋₁₈ alkyl, C₂₋₁₈ alkenyl, C₂₋₁₈ alkynyl, C₅₋₁₀ aryl, C₁₋₁₈ heteroalkyl, C₅₋₁₈ heteroaryl, C₃₋₁₂ cycloalkyl, C₃₋₁₂ heterocyclyl, -OC₁₋₁₈ alkyl is optionally substituted with one or more substituents selected from NR₃R₄, -N⁺R₃R₄R₅, -OC₁₋₁₈ alkyl, hydroxyl, cyano, C₁₋₆ alkoxy, C₁₋₆ haloalkyl, C₃₋₆ cycloalkyl, C₅₋₁₀ aryl, C₃₋₆ heterocyclyl, or C₅₋₆ heteroaryl;
R₁ is selected from hydrogen, C₁₋₁₈ alkyl, C₂₋₁₈ alkenyl, C₂₋₁₈ alkynyl, C₅₋₁₀ aryl, C₁₋₁₈ heteroalkyl, C₅₋₁₈ heteroaryl, C₃₋₁₂ cycloalkyl, C₃₋₁₂ heterocyclyl, NR₃R₄, -N⁺R₃R₄R₅, -OC₁₋₁₈alkyl, amino acids, peptides, nucleobases, sugars, lipids, or -COOH, wherein C₁₋₁₈ alkyl, C₂₋₁₈ alkenyl, C₂₋₁₈ alkynyl, C₅₋₁₀ aryl, C₁₋₁₈ heteroalkyl, C₅₋₁₈ heteroaryl, C₃₋₁₂ cycloalkyl, C₃₋₁₂ heterocyclyl, OC₁₋₁₈ alkyl is optionally substituted with 5-10 membered monocyclic or bicyclic aryl optionally substituted with 1-5 substituents selected from NR₃R₄, hydroxyl, cyano, halogen, C₁₋₁₈ alkyl, C₁₋₁₈ alkoxy, C₃₋₁₂ cycloalkyl, C₁₋₁₈ haloalkyl, C₁₋₁₈ haloalkoxy, C₁₋₁₈ acylamino, or C₁₋₁₈ alkylamino; and R₃, R₄, R₅ is independently selected from hydrogen or C₁₋₁₈ alkyl.

In a second aspect of the present disclosure, there is provided a process of preparation of compound of Formula (I), and its polymorphs, stereoisomers, prodrugs, solvates, co-crystals, intermediates, pharmaceutically acceptable salts, and metabolites thereof, the process comprising reacting:

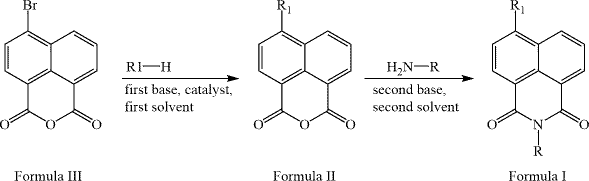

wherein R is selected from C₁₋₁₈ alkyl, C₂₋₁₈ alkenyl, C₂₋₁₈ alkynyl, C₅₋₁₀ aryl, C₁₋₁₈ heteroalkyl, C₅₋₁₈ heteroaryl, C₃₋₁₂ cycloalkyl, C₃₋₁₂ heterocyclyl, NR₃R₄, -N⁺R₃R₄R₅, -OC₁₋₁₈ alkyl, amino acids, peptides, nucleobases, sugars, or lipids, wherein C₁₋₁₈ alkyl, C₂₋₁₈ alkenyl, C₂₋₁₈ alkynyl, C₅₋₁₀ aryl, C₁₋₁₈ heteroalkyl, C₅₋₁₈ heteroaryl, C₃₋₁₂ cycloalkyl, C₃₋₁₂ heterocyclyl, -OC₁₋₁₈alkyl is optionally substituted with one or more substituents selected from -NR₃R₄, -N⁺R₃R₄R₅, -OC₁₋₁₈ alkyl, hydroxyl, cyano, C₁₋₆ alkoxy, C₁₋₆ haloalkyl, C₃₋₆ cycloalkyl, C₅₋₁₀ aryl, C₃₋₆ heterocyclyl, or C₅₋₆ heteroaryl, wherein-OC₁₋₁₈ alkyl, C₁₋₆ alkoxy, C₁₋₆ haloalkyl, C₃₋₆ cycloalkyl, C₅₋₁₀ aryl, C₃₋₆ heterocyclyl, or C₅₋₆ heteroaryl is further substituted with hydroxyl, cyano, C₁₋₆ alkoxy, C₁₋₆ haloalkyl, C₃₋₆ cycloalkyl, C₅₋₁₀ aryl, C₃₋₆ heterocyclyl, or C₅₋₆ heteroaryl;
R₁ is selected from hydrogen, C₁₋₁₈ alkyl, C₂₋₁₈ alkenyl, C₂₋₁₈ alkynyl, C₅₋₁₀ aryl, C₁₋₁₈ heteroalkyl, C₅₋₁₈ heteroaryl, C₃₋₁₂ cycloalkyl, C₃₋₁₂ heterocyclyl, -NR₃R₄, -N⁺R₃R₄R₅, -OC₁₋₁₈ alkyl, amino acids, peptides, nucleobases, sugars, lipids, or -COOH, wherein C₁₋₁₈ alkyl, C₂₋₁₈ alkenyl, C₂₋₁₈ alkynyl, C₅₋₁₀ aryl, C₁₋₁₈ heteroalkyl, C₅₋₁₈ heteroaryl, C₃₋₁₂ cycloalkyl, C₃₋₁₂ heterocyclyl, OC₁₋₁₈ alkyl is optionally substituted with 5-10 membered monocyclic or bicyclic aryl optionally substituted with 1-5 substituents selected from NR₃R₄, hydroxyl, cyano, halogen, C₁₋₁₈ alkyl, C₁₋₁₈ alkoxy, C₃₋₁₂ cycloalkyl, C₁₋₁₈ haloalkyl, C₁₋₁₈ haloalkoxy, C₁₋₁₈ acylamino, or C₁₋₁₈ alkylamino; and R₃, R₄, and R₅ are independently selected from hydrogen or C₁₋₁₈ alkyl.

In a third aspect of the present disclosure, there is provided a pharmaceutical composition comprising the compound of Formula (I) or a pharmaceutically acceptable salt thereof with a pharmaceutically acceptable carrier, optionally in combination with one or more other pharmaceutical compositions.

In a fourth aspect of the present disclosure, there is provided a method for the treatment of a condition mediated by a neurodegenerative disease, said method comprising administering to a subject an effective amount of the compound of Formula (I) or the pharmaceutical composition as described herein.

In a fifth aspect of the present disclosure, there is provided a method of treatment of a condition mediated by a neurodegenerative disease, said method comprising administering a combination of the compound of Formula (I) or the pharmaceutical composition as described herein with other clinically relevant immune modulator agents to a subject in need of thereof.

In a sixth aspect of the present disclosure, there is provided a use of the compound of Formula (I) or the pharmaceutical composition as described herein for treatment of a condition mediated by aggregation of Aβ42, tau, or α-syn .

In a seventh aspect of the present disclosure, there is provided a use of the compound of Formula (I) or the pharmaceutical composition as described herein with other clinically relevant agents or biological agents for the treatment of a condition mediated by aggregation of Aβ42, tau, or α-syn.

These and other features, aspects, and advantages of the present subject matter will be better understood with reference to the following description. This summary is provided to introduce a selection of concepts in a simplified form. This summary is not intended to identify key features or essential features of the claimed subject matter.

### BRIEF DESCRIPTION OF ACCOMPANYING DRAWINGS

The following drawings form a part of the present specification and are included to further illustrate aspects of the present disclosure. The disclosure may be better understood by reference to the drawings in combination with the detailed description of the specific embodiments presented herein.

FIG. 1(a) depicts fluorescence intensity (NFI) of ThT at 482 nm for 1:1 samples of TGR63-68, in accordance with an embodiment of the present disclosure.

FIG. 1(b) depicts ThT binding assay in terms of % inhibition for 1:1, 1:2, and 1:5 samples each of TGR63-65, in accordance with an embodiment of the present disclosure.

FIG. 1(c) depicts ThT binding assay in terms of % dissolution for 1:1, 1:2, and 1:5 samples each of TGR63-65, in accordance with an embodiment of the present disclosure.

FIG. 2 depicts cellular viability in terms of cell viability % for 1:2 samples of TGR63-68, in accordance with an embodiment of the present disclosure.

FIG. 3 depicts ¹H NMR spectra of TGR63, in accordance with an embodiment of the present disclosure.

FIG. 4(a) depicts TEM images of Aβ42 aggregation species in the absence of TGR63, in accordance with an embodiment of the present disclosure.

FIG. 4(b) depicts TEM images of Aβ42 aggregation species in the presence of TGR63, in accordance with an embodiment of the present disclosure.

FIG. 4(c) depicts dot blot images on PVDF membrane of Aβ42 fibrils done using OC antibody, L1: absence of TGR63; L2: presence of 10 µM TGR63; L3: presence of 50 µM TGR63, in accordance with an embodiment of the present disclosure.

FIG. 4(d) depicts quantification results of Aβ fibrils corresponding to L1. L2 and L3 as described in FIG. 4(c), in accordance with an embodiment of the present disclosure.

FIG. 5 depicts images for membrane toxicity modulation ability of TGR63 obtained by nuclear staining of SHSY5Y cells with DAPI (blue) and Aβ42 fibrils staining with OC antibody and red fluorescent labelled secondary antibody, in accordance with an embodiment of the present disclosure.

FIG. 6 depicts ThT binding assay in terms of % inhibition for 1:1, 1:2, and 1:5 samples of each TGR63 and TGR64 with a fixed concentration of α-Syn at 100 µM (characteristic of Parkinson’s disease), in accordance with an embodiment of the present disclosure.

FIG. 7 (a) depicts the calculation of lethal dose 50% (LD50) of TGR63 through intraperitoneal administration, the experimental details and the final observation on 14th day; (b) depicts the plot of mortality (%) against TGR63 concentration and calculation of LD50, in accordance with an embodiment of the present disclosure.

FIG. 8 depicts the MALDI mass analysis of vehicle (9A) and TGR63 treated mice blood serum after 1 h (9B) and 24 h (9C) of administration, in accordance with an embodiment of the present disclosure.

FIG. 9 depict the serum stability of TGR63 under in vitro conditions: TGR63 was incubated in PBS (10 mM, pH= 7.4) and blood serum (WT mouse) for different time (0.5, 1, 2 and 6 h) at 37° C., in accordance with an embodiment of the present disclosure.

FIG. 10A depicts the Calculation of LogP Standard concentration curve obtained by measuring absorbance at 480 nm for 1, 5, 10, 20 and 50 µM of TGR63 in octanol, and FIG. 10B Absorbance of octanol layer (Sample_Octanol) and calculation of LogP, in accordance with an embodiment of the present disclosure.

FIG. 11 depicts MALDI mass analysis of vehicle (12A) and TGR63 (12B) treated mouse brain lysate after 1 h, in accordance with an embodiment of the present disclosure.

FIG. 12 depicts the evaluation of organ toxicity of TGR63, bright field images of vehicle and TGR63 treated mice organs (liver, heart, spleen and kidney) stained with hematoxylin and eosin, in accordance with an embodiment of the present disclosure.

FIG. 13 depicts the staining of amyloid plaques with OC primary antibody and ThT or CQ probe: (13A) The high resolution confocal microscopy images of cortex and hippocampus regions of the AD mouse brain, immunostained with OC antibody (red), DAPI (blue) and ThT (green). The merged images display significant overlap between ThT and OC staining to confirm the amyloid deposition (pointed with white arrows). (13B) Visualization of amyloid deposits associated neuronal damage, in accordance with an embodiment of the present disclosure.

FIG. 14 depicts the experimental planning and TGR63 administration in APP/PS1 mice (age in month, m), in accordance with an embodiment of the present disclosure.

FIG. 15A represents the visualization of amyloid plaques in half hemisphere; 15B illustrates the reduction of cortical and hippocampal amyloid burden by TGR63 treatment; 15C and 15D depicts the quantification of Aβ plaques i.e., amount of Aβ plaques (% area) deposited in different regions (cortex and hippocampus) of vehicle and TGR63 treated mice (WT and AD) brain, in accordance with an embodiment of the present disclosure.

FIG. 16A depicts the improvement of memory and cognitive functions by TGR63 in APP/PS1 AD phenotypic mice via tracing of vehicle and TGR63 treated mice (WT and AD) locomotion during open field (OF) test (test period: 5 min); FIG. 16B depicts the total distance traveled by experimental mice cohorts; FIG. 16C depicts the average number of entries into the center zone; FIG. 16D depicts the distance traveled by experimental mice cohorts in the center zone; FIG. 16E depicts the novel object identification (NOI) test protocol by capturing the image of experimental arenas during habituation, familiarization and test days; FIGS. 16F and 16G depicts the recognition of novel objects compared to old object on test day 1 and 2, respectively; FIG. 16H depicts the Morris water maze (MWM) test analysis; FIG. 16I depicts the latency time (second) of each cohort for searching the hidden platform during training; FIG. 16J depicts the representative trace of experimental mouse in probe trail (no platform); FIG. 16K depicts the percentage of total exploration by each cohort in target quadrant (platform was placed during training) and other quadrants in probe trial; FIG. 16L depicts the average number of target (platform) crossing by each cohort during probe trail (no platform), in accordance with an embodiment of the present disclosure.

FIG. 17 depicts the locomotion of vehicle treated WT mice cohort during OF test, in accordance with an embodiment of the present disclosure.

FIG. 18 depicts the locomotion of TGR63 treated WT mice cohort during OF test, in accordance with an embodiment of the present disclosure.

FIG. 19 depicts the locomotion of vehicle treated AD mice cohort during OF test, in accordance with an embodiment of the present disclosure.

FIG. 20 depicts the locomotion of TGR63 treated AD mice cohort during OF test, in accordance with an embodiment of the present disclosure.

FIG. 21 depicts the trajectory of vehicle treated WT mice cohort during MWM probe trail (without platform), in accordance with an embodiment of the present disclosure.

FIG. 22 depicts the trajectory of TGR63 treated WT mice cohort during MWM probe trail (without platform), in accordance with an embodiment of the present disclosure.

FIG. 23 depicts the trajectory of vehicle treated AD mice cohort during MWM probe trail (without platform), in accordance with an embodiment of the present disclosure.

FIG. 24 depicts the trajectory of TGR63 treated AD mice cohort during MWM probe trail (without platform), in accordance with an embodiment of the present disclosure.

FIG. 25 depicts the inhibition of tau (5 µM) aggregation in presence of TGR63, in accordance with an embodiment of the present disclosure.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

The term “at least one” used herein refers to one or more and thus includes individual components as well as mixtures/combinations.

The term “polymorphs” refers to crystal forms of the same molecule, and different polymorphs may have different physical properties such as, for example, melting temperatures, heats of fusion, solubilities, dissolution rates and/or vibrational spectra as a result of the arrangement or conformation of the molecules in the crystal lattice. It will be further appreciated that certain compounds of the invention that exist in crystalline form, including the various solvates thereof, may exhibit polymorphism (i.e. the capacity to occur in different crystalline structures). These different crystalline forms are typically known as ‘polymorphs’. The invention includes such polymorphs. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. It will be appreciated that different polymorphs may be produced, for example, by changing or adjusting the reaction conditions or reagents, used in making the compound. For example, changes in temperature, pressure, or solvent may result in polymorphs. In addition, one polymorph may spontaneously convert to another polymorph under certain conditions.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described hereinabove. The permissible substituents can be one or more and the same or different for appropriate organic compounds. For purposes of this disclosure, the heteroatoms such as nitrogen may have hydrogen substituents, and/or any permissible substituents of organic compounds described herein which satisfy the valences of the heteroatoms.

The term “solvate”, as used herein, refers to a crystal form of a substance which contains solvent.

The term “hydrate” refers to a solvate wherein the solvent is water.

The term “prodrug” refers to a derivative of a drug molecule as, for example, esters, carbonates, carbamates, urea, amides or phosphates that requires a transformation within the body to release the active drug. Prodrugs are frequently, although not necessarily, pharmacologically inactive until converted to the parent drug. Prodrugs may be obtained by bonding a pro-moiety (defined herein) typically via a functional group, to a drug. Some examples of prodrugs within the scope of this invention include: if the compound contains a hydroxyl group, the hydroxyl group may be modified to form an ester, carbonate, or carbamate. Examples include acetate, pivalate, methyl and ethyl carbonates, and dimethylcarbamate. The ester may also be derived from amino acids such as glycine, serine. or lysine. If the compound contains an amine group, the amine group may be modified to form an amide. Examples include acetamide or derivatization with amino acids such as glycine, serine. or lysine.

The term “co-crystal” refers to crystalline phase materials made of two or more components, wherein the components may be atoms, ions, or molecules.

The term “intermediate” refers to all those molecules that occur in between the reaction pathway and share common structural features with the compounds of the present disclosure.

The term “pharmaceutically acceptable salt” embraces salts with a pharmaceutically acceptable acid or base. Pharmaceutically acceptable acids include both inorganic acids, for example hydrochloric, sulphuric, phosphoric, diphosphoric, hydrobromic, hydroiodic and nitric acid and organic acids, for example citric, fumaric, maleic, malic, mandelic, ascorbic, oxalic, succinic, tartaric, benzoic, acetic, methane sulphonic, ethane sulphonic, benzene sulphonic or p-toluene sulphonic acid. Pharmaceutically acceptable bases include alkali metal (e.g. sodium or potassium) and alkali earth metal (e.g. calcium or magnesium) hydroxides and organic bases, for example alkyl amines, arylalkyl amines and heterocyclic amines.

The term “metabolites” refers to all those chemical compounds that are necessary for the metabolism or are formed during the metabolic pathway of a cell.

Other preferred salts according to the invention are quaternary ammonium compounds wherein an equivalent of an anion (X-) is associated with the positive charge of the N atom. X- may be an anion of various mineral acids such as, for example, chloride, bromide, iodide, sulphate, nitrate, phosphate, or an anion of an organic acid such as, for example, acetate, maleate, fumarate, citrate, oxalate, succinate, tartrate, malate, mandelate, trifluoroacetate, methane sulphonate and p-toluene sulphonate. X-is preferably an anion selected from chloride, bromide, iodide, sulphate, nitrate, acetate, maleate, oxalate, succinate or trifluoroacetate. More preferably X- is chloride, bromide, trifluoroacetate or methane sulphonate. Nonlimiting examples of pharmaceutically acceptable salts include but are not limited to glycolate, fumarate, mesylate, cinnamate, isethionate, sulfate, phosphate, diphosphate, nitrate, hydrobromide, hydroiodide, succinate, formate, acetate, dichloroacetate, lactate, p-toluenesulfonate, pamitate, pidolate. pamoate, salicylate, 4-aminosalicylate, benzoate, 4-acetamido benzoate, glutamate, aspartate, glycolate, adipate, alginate, ascorbate, besylate, camphorate, camphorsulfonate, camsylate, caprate, caproate, cyclamate, laurylsulfate, edisylate, gentisate, galactarate, gluceptate, gluconate, glucuronate, oxoglutarate, hippurate, lactobionate, malonate, maleate, mandalate, napsylate, napadisylate, oxalate, oleate, sebacate, stearate, succinate, thiocyanate, undecylenate, and xinafoate.

The term “effective amount” means an amount of a compound or composition which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of the treatment, the nature of concurrent therapy, the particular active ingredient(s) being employed, the particular pharmaceutically-acceptable excipient(s)/carrier(s) utilized, the route of administration, and like factors within the knowledge and expertise of the attending physician.

Compounds of the present invention may be combined with a pharmaceutically acceptable carrier to provide pharmaceutical compositions useful for treating the conditions or disorders. The particular carrier employed in the pharmaceutical compositions may vary depending upon the type of administration desired (e.g. intravenous, oral, topical, suppository, or parenteral). For example, in preparing the compositions in oral liquid dosage forms (e.g. suspensions, elixirs and solutions), typical pharmaceutical media include but not limited to water, glycols, oils, alcohols, flavoring agents, preservatives, coloring agents and the like. Similarly, for preparing oral solid dosage forms (e.g. powders, tablets and capsules), carriers include but not limited to starches, sugars, diluents, granulating agents, lubricants, binders, disintegrating agents and the like.

Typical compositions include a compound of the invention and a pharmaceutically acceptable carrier. For example, the active compound will be mixed with a carrier, or diluted by a carrier, or enclosed within a carrier which can be in the form of an ampoule, capsule, sachet, paper, or other container. When the compound is mixed with a carrier, or when the carrier serves as a diluent, it can be solid, semi-solid, or liquid material that acts as a vehicle, excipient, or medium for the active compound. The compound can be adsorbed on a granular solid carrier, for example contained in a sachet. Some examples of suitable carriers include but not limited to water, salt solutions, alcohols, polyethylene glycols. polyhydroxyethoxylated castor oil, peanut oil, olive oil, gelatin, lactose, terra alba, sucrose, dextrin. magnesium carbonate, sugar, cyclodextrin, amylose, magnesium stearate, talc, agar, pectin, acacia, stearic acid or lower alkyl ethers of cellulose, silicic acid, fatty acids, fatty acid amines, fatty acid mono glycerides and diglycerides, pentaerythritol fatty acid esters, polyoxyethylene. hydroxymethylcellulose, and polyvinylpyrrolidone. Similarly, the carrier or diluent can include any sustained release material known in the art such as glyceryl monostearate or glyceryl distearate, alone or mixed with a wax.

The term “one or more other pharmaceutical composition” refers to other active pharmaceutical ingredients or composition that can work in combination with pharmaceutical composition of the present disclosure. The other pharmaceutical composition includes but not limited to Food and Drug Administration (FDA) approved drugs for the preliminary medication of AD patients and inflammation such as Aricept® (donepezil), Exelon® (rivastigmine), Razadyne® (galantamine), Namenda® (memantine), Nonsteroidal anti-inflammatory drugs (NSAIDs).

In this specification, the prefix C_{x-y} as used in terms such as C_{x-y} alkyl and the like (where x and y are integers) indicates the numerical range of carbon atoms that are present in the group; for example, C₁₋₁₈ alkyl includes C₃ alkyl (propyl and isopropyl), C₄ alkyl (butyl, 1-methylpropyl, 2-methylpropyl, and t-butyl), and the like. Unless specifically stated, the bonding atom of a group may be any suitable atom of that group; for example, propyl includes prop-1-yl and prop-2-yl.

The term “C₁₋₁₈ alkyl” as used herein refers to a radical or group which may be saturated or unsaturated, linear or branched hydrocarbons, unsubstituted or mono- or poly-substituted.

The term “alkyl” refers to a mono-radical, branched or unbranched, saturated hydrocarbon chain having from 1 to 18 carbon atoms. This term is exemplified by groups such as n-butyl, iso-butyl, t-butyl, n-hexyl, and the like. The groups may be optionally substituted.

The term “alkenyl” refers to a mono-radical of a branched or unbranched unsaturated hydrocarbon group preferably having from 2, 3, 4, 5, to 18 carbon atoms and having 1, 2, 3, inter alia double bonds. The groups may be optionally substituted.

The term “heteroalkyl” refers to an alkyl radical having 1 to 18 carbon atoms and one or more skeletal carbon atoms replaced by heteroatoms selected from oxygen, nitrogen and sulfur. The alkyl chain may be optionally substituted.

The term “heteroaryl” refers to an aromatic cyclic group having 5, 6, 7, 8, 9, 10, 11, 12, 13, 14, 15, 16, 17 or 18 carbon atoms and 1, 2, 3 or 4 heteroatoms selected from oxygen, nitrogen and sulfur within at least one ring. Such heteroaryl groups can have a single ring (e.g. pyridyl or furyl) or multiple condensed rings (e.g. indolizinyl, benzothiazolyl, or benzothienyl). Examples of heteroaryls include, but are not limited to, [1,2,4] oxadiazole, [1,3,4] oxadiazole, [1,2,4] thiadiazole, [1,3,4] thiadiazole, pyrrole, imidazole, pyrazole, pyridine, pyrazine, pyrimidine, pyridazine, indolizine, isoindole, indole, indazole, purine, quinolizine, isoquinoline, quinoline, phthalazine, quinoxaline, quinazoline, cinnoline, pteridine, carbazole, carboline, phenanthridine, acridine, phenanthroline, isothiazole, phenazine, isoxazole, phenoxazine, phenothiazine, furan, thiophene, oxazole, thiazole, triazole, triazine and the like.

The term “heterocyclyl” refers to a saturated or partially unsaturated group having a single ring or multiple condensed rings, having from 3 to 12 carbon atoms and from 1 to 10 hetero atoms, preferably 1, 2, 3 or 4 heteroatoms, selected from nitrogen, sulfur, phosphorus, and/or oxygen within the ring. Heterocyclic groups can have a single ring or multiple condensed rings, and include tetrahydrofuranyl, morpholinyl, piperidinyl, piperazinyl, dihydropyridinyl, tetrahydroquinolinyl and the like. The groups may be optionally substituted.

The term “alkynyl” refers to a branched or unbranched, unsaturated chain of carbon atoms having 2 to 18 carbon atoms and one or more than one carbon-carbon triple bonds. The groups may be optionally substituted.

The term “hydroxyl” refers to an —OH moiety attached to a main chain of carbon atoms.

The term “cyano” refers to the group —CN attached to a main chain of carbon atoms.

The term “alkoxy” refers to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O—\alkyl, —O— alkenyl, \—O—\alkynyl.

The term “haloalkyl” refers to an alkyl radical having 1 to 6 carbon atoms and one or more skeletal carbon atoms replaced by halogens selected from fluorine, chlorine, bromide, or iodine.

The term “haloalkoxy” refers to —O—\haloalkyl group where the haloalkyl group has an alkyl radical having 1 to 6 carbon atoms and one or more skeletal carbon atoms replaced by halogens selected from fluorine, chlorine, bromide, or iodine. The alkyl chain may be optionally substituted.

The term “acylamino” refers to the group —NR″C(O)R′ wherein each R′ and R″ can be branched or unbranched, saturated or unsaturated, chain of carbon atoms where acyl group has 1 to 18 carbon atoms. The groups may be optionally substituted.

The term “alkylamino” refers to amino group, to which a straight chain or branched chain alkyl group with 1 to 18 carbon atoms is bound. Representative examples of alkylamino include but are not limited to methylamino, ethylamino, propylamino, butylamino and the like.

The term “alkyl amine” refers to a group having alkyl attached to an amine. The alkyl amine may include primary, secondary or tertiary amine. In the present disclosure, alkyl amine comprises carbon ranging between 1 to 10 atoms with an amine group. The alkyl group may be saturated or unsaturated.

The term “halogen” refers to fluorine, chlorine, bromide, or iodine.

The term “cycloalkyl” refers to carbocyclic groups of from 3 to 12 carbon atoms having a single cyclic ring or multiple condensed rings which may be partially unsaturated. Such cycloalkyl groups include, by way of example, single ring structures such as cyclopropyl, cyclobutyl, cyclopentyl, cyclopentenyl, cyclohexyl, cyclohexenyl, and the like, or multiple ring structures or carbocyclic groups to which is fused an aryl group, for example indane, and the like. The groups may be optionally substituted.

The term “aryl” refers to any mono- and poly-carbocyclic ring systems wherein the individual carbocyclic rings in the polyring systems are fused or attached to each other via a single bond and wherein at least one ring is aromatic. Unless otherwise indicated, substituents to the aryl ring systems can be attached to any ring atom, such that the attachment results in formation of a stable ring system.

The term “amino acids” refers to an organic compound comprising both carboxyl group and amino group. It may be further specified as modified or unmodified natural or unnatural amino acids which includes but not limited to 1-dopamine, Fmoc-L -glutamic acid 5-tert-butyl ester (Fmoc-Glu(OtBu)-OH), histidine, isoleucine, leucine, lysine, methionine, phenylalanine, threonine, tryptophan, valine, citrulline, hydroxyproline, norleucine, 3-nitrotyrosine, nitroarginine, ornithine, naphtylalanine, methionine sulfoxide, or methionine sulfone.

The term “peptides” refers to compounds comprising two or more amino acids. Peptides can be classified as dipeptides, tripeptides, and tetrapeptides, oligopeptides, or polypeptides based on number of amino acids present in the peptide. The term peptide also includes peptidomimetic which refers to small protein-like chain designed to mimic a peptide. They typically arise either from modification of an existing peptide, or by designing similar systems that mimic peptides, such as peptoids and β-peptides. Examples of peptides include but not limited to Glycine-Histidine-Lysine (GHK), cyclic-dipeptides, peptidomimetics, various fragments of APP, α-Syn and Tau proteins.

The term “nucleobases” refers to nitrogenous bases or their derivatives, that are nitrogen-containing biological compounds that form nucleosides, which, in turn, are components of nucleotides, with all of these monomers constituting the basic building blocks of nucleic acids. Examples of nucleobases includes but not limited to adenine (A), cytosine (C), guanine (G), thymine (T), uracil (U) and their derivatives.

The term “sugars” refers to compounds of aldoses or ketoses. Examples of sugars includes but not limited to glyceraldehyde, erythrose, threose, ribose, arabinose, xylose, lyxose, allose, altrose, glucose, mannose, gulose, idose, galactose and talose.

The term “lipids” refers to organic compounds that are fatty acids or their derivatives and are insoluble in water but soluble in organic solvents. Lipids of the present disclosure may be selected from sterol derivatives, prenol derivatives, fatty acid derivatives (C₄₋₂₈). Examples of lipids includes but not limited to Cholesterol, Ergosterol, Hopanoids, Hydroxysteroid, Phytosterol, Steroids, Zoosterol, saturated and unsaturated fatty acids.

The term “Aβ42” refers to amyloid-β peptide produced in the brain and is a 42 amino acid proteolytic product from the amyloid precursor protein. The peptide is considered as a biomarker for correlating with Alzheimer disease (AD) onset, mild cognitive impairment, vascular dementia, and other cognitive disorders.

The term “tau” refers to a class of microtubule-associated protein (MAP), helping to maintain and stabilize the microtubule assembly in matured neurons. Tau interacts with tubulin and stimulates its assembly into microtubules to maintain structure and function of neuronal cells. The self-aggregation of hyperphosphorylated Tau form intracellular neurofibrillary tangles and paired helical filaments that is associated with the onset of neurodegenerative disorders like AD and taupathies.

The term “α-syn” refers to Alpha-synuclein, a small presynaptic protein that encoded by the SNCA gene in human. Alpha-synuclein regulates synaptic vesicle trafficking and subsequent neurotransmitter release. The misfolding of Alpha-synuclein into β-sheet secondary structure is mainly responsible for pathogenic α-Syn aggregation and LB (lewy body) formation, which further leads to neurodegenerative disorder like Parkinson disease. α-syn is also reported to play a role in AD.

A term once described, the same meaning applies for it, throughout the disclosure.

The compound of Formula (I), and its polymorphs, stereoisomers, prodrugs, solvates, co-crystals, intermediates, pharmaceutically acceptable salts, and metabolites thereof can also be referred as “compounds of the present disclosure” or “compounds”.

Furthermore, the compound of Formula (I), can be its derivatives, analogs, stereoisomer’s, diastereomers, geometrical isomers, polymorphs, solvates, co-crystals, intermediates, hydrates, metabolites, prodrugs or pharmaceutically acceptable salts and compositions.

It is understood that included in the family of compounds of Formula (I), are isomeric forms including diastereoisomers, enantiomers, tautomers, and geometrical isomers in “E” or “Z” configurational isomer or a mixture of E and Z isomers. It is also understood that some isomeric forms such as diastereomers, enantiomers and geometrical isomers can be separated by physical and/or chemical methods by those skilled in the art.

Compounds disclosed herein may exist as single stereoisomers, racemates and or mixtures of enantiomers and/or diastereomers. All such single stereoisomers, racemates and mixtures thereof are intended to be within the scope of the subject matter described.

Compounds disclosed herein include isotopes of hydrogen, carbon, oxygen, fluorine, chlorine, iodine and sulfur which can be incorporated into the compounds, such as, but not limited to, ²H (D), ³H (T), ¹¹C, ¹³C, ¹⁴C, ¹⁵N, ¹⁸F, ³⁵S, ³⁶Cl, and ¹²⁵I. Compounds of this disclosure wherein atoms were isotopically labeled for example radioisotopes such as ³H, ¹³C, ¹⁴C, and the like can be used in metabolic studies, kinetic studies, and imaging techniques such as positron emission tomography used in understanding the tissue distribution of the drugs. Compounds of the disclosure where hydrogen is replaced with deuterium may improve the metabolic stability, and pharmacokinetics properties of the drug such as in vivo half-life.

As it is discussed in the background, many of the neurodegenerative disorders, among other pathogenesis, majorly arise due to amyloidogenic toxicity. According to the amyloid hypothesis, the aggregation-prone monomeric Aβ42 readily self-assembles themselves in the ordered β-sheet structure mostly through non-covalent interactions such as hydrogen bonding, hydrophobic and electrostatic interactions. Contemporary studies have also revealed that the aggregated Aβ species bind with plasma membrane and prompt the internalization of misfolded Aβ peptides, which finally lead to the irrecoverable amyloid toxicity. The existing therapeutics reveal various classes of molecules like peptides, peptidomimetics, polymers, and synthetic compounds which have been extensively evaluated as modulators against protein aggregation, with little or no success due to sluggish therapeutic action, ineffectiveness for the treatment at moderate and advanced stages of the disease, low natural abundance, poor solubility, instability and most importantly lack of multifunctional efficacy in targeting multifaceted Aβ toxicity.

In view of this, the present disclosure discloses small molecule-based naphthalene monoimide (NMI) compounds that can act as a core hydrophobic platform for better and stronger interaction with hydrophobic pockets of amyloid fibrils. With the huge scope of further synthetic modifications, bromo-naphthalene monoanhydride can be conjugated to N,N-dimethylaniline at the 8^{th} position through an alkynyl linker using sonogashira coupling reaction. N,N-Dimethylaniline conjugated to naphthalene monoanhydride was believed to provide a balanced hydrophobicity to improve the aggregation inhibition property. However, hydrophobic nature of naphthalene monoanhydride derivatives poses solubility issues during in cellulo and in vivo experiments. To address this issue, further modifications of these naphthalene monoanhydride derivatives with N,N,N-trimethylethylenediamine, ethylenediamine, 2-(2-aminoethoxy) ethanol, among others, can be carried out to obtain the NMI compounds of the present disclosure, that are compatible with the hydrophobic Aβ and α-syn aggregates, that are potent amyloidogenesis inhibitors, and useful in the treatment of various disease states related to amyloidogenic toxicity, for example, Alzheimer’s diseases and Parkinson’s disease.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described. All publications mentioned herein are incorporated herein by reference.

The present disclosure is not to be limited in scope by the specific embodiments described herein, which are intended for the purposes of exemplification only. Functionally-equivalent products, compositions, and methods are clearly within the scope of the disclosure, as described herein.

In an embodiment of the present disclosure, there is provided a compound of Formula (I)

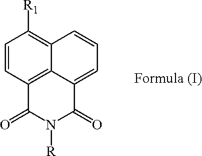

and its polymorphs, stereoisomers, prodrugs, solvates, co-crystals, intermediates, pharmaceutically acceptable salts, and metabolites thereof, wherein
R is selected from C₁₋₁₈ alkyl, C₂₋₁₈ alkenyl, C₂₋₁₈ alkynyl, C₅₋₁₀ aryl, C₁₋₁₈ heteroalkyl, C₅₋₁₈ heteroaryl, C₃₋₁₂ cycloalkyl, C₃₋₁₂ heterocyclyl, -NR₃R₄, -N⁺R₃R₄R₅, -OC₁₋₁₈ alkyl, amino acids, peptides, nucleobases, sugars, or lipids, wherein C₁₋₁₈ alkyl, C₂₋₁₈ alkenyl, C₂₋₁₈ alkynyl, C₅₋₁₀ aryl, C₁₋₁₈ heteroalkyl, C₅₋₁₈ heteroaryl, C₃₋₁₂ cycloalkyl, C₃₋₁₂ heterocyclyl, -OC₁₋₁₈ alkyl is optionally substituted with one or more substituents selected from -NR₃R₄, -N⁺R₃R₄R₅, -OC₁₋₁₈ alkyl, hydroxyl, cyano, C₁₋₆ alkoxy, C₁₋₆ haloalkyl, C₃₋₆ cycloalkyl, C₅₋₁₀ aryl, C₃₋₆ heterocyclyl, or C₅₋₆ heteroaryl, wherein -OC₁₋₁₈ alkyl, C₁₋₆ alkoxy, C₁₋₆ haloalkyl, C₃₋₆ cycloalkyl, C₅₋₁₀ aryl, C₃₋₆ heterocyclyl, or C₅₋₆ heteroaryl is further substituted with hydroxyl, cyano, C₁₋₆ alkoxy, C₁₋₆ haloalkyl, C₃₋₆ cycloalkyl, C₅₋₁₀ aryl, C₃₋₆ heterocyclyl, or C₅₋₆ heteroaryl;
R₁ is selected from hydrogen, C₁₋₁₈ alkyl, C₂₋₁₈ alkenyl, C₂₋₁₈ alkynyl, C₅₋₁₀ aryl, C₁₋₁₈ heteroalkyl, C₅₋₁₈ heteroaryl, C₃₋₁₂ cycloalkyl, C₃₋₁₂ heterocyclyl, -NR₃R₄, -N⁺R₃R₄R₅, -OC₁₋₁₈ alkyl, amino acids, peptides, nucleobases, sugars, lipids, or -COOH, wherein C₁₋₁₈ alkyl, C₂₋₁₈ alkenyl, C₂₋₁₈ alkynyl, C₅₋₁₀ aryl, C₁₋₁₈ heteroalkyl, C₅₋₁₈ heteroaryl, C₃₋₁₂ cycloalkyl, C₃₋₁₂ heterocyclyl, OC₁₋₁₈ alkyl is optionally substituted with 5-10 membered monocyclic or bicyclic aryl optionally substituted with 1-5 substituents selected from -NR₃R₄, hydroxyl, cyano, halogen, C₁₋₁₈ alkyl, C₁₋₁₈ alkoxy, C₃₋₁₂ cycloalkyl, C₁₋₁₈ haloalkyl, C₁₋₁₈ haloalkoxy, C₁₋₁₈ acylamino, or C₁₋₁₈ alkylamino; and R₃, R₄, and R₅ are independently selected from hydrogen or C₁₋₁₈ alkyl.

In an embodiment of the present disclosure, there is provided a compound of Formula (I), and its polymorphs, stereoisomers, prodrugs, solvates, co-crystals, intermediates, pharmaceutically acceptable salts, and metabolites thereof, wherein R is C₁₋₁₈ alkyl optionally substituted with one or more substituents selected from -NR₃R₄, -N⁺R₃R₄R₅, -OC₁₋₆ alkyl, hydroxyl, cyano, C₁₋₆ alkoxy, C₁₋₆ haloalkyl, C₃₋₆ cycloalkyl, C₅₋₁₀ aryl, C₃₋₆ heterocyclyl, C₅₋₆ heteroaryl, amino acids, peptides, nucleobases, sugars, or lipids, and wherein -OC₁₋₆ alkyl, C₁₋₆ alkoxy, C₁₋₆ haloalkyl, C₃₋₆ cycloalkyl, C₅₋₁₀ aryl, C₃₋₆ heterocyclyl, or C₅₋₆ heteroaryl is further substituted with hydroxyl, cyano, C₁₋₆ alkoxy, C₁₋₆ haloalkyl, C₃₋₆ cycloalkyl, C₅₋₁₀ aryl, C₃₋₆ heterocyclyl, or C₅₋₆ heteroaryl;
R₁ is selected from hydrogen, -NR₃R₄, C₂₋₆ alkynyl, amino acids, peptides, nucleobases, sugars, or lipids, wherein C₂₋₆ alkynyl is optionally substituted with 5-10 membered monocyclic or bicyclic aryl optionally substituted with 1-5 substituents selected from -NR₃R₄, hydroxyl, cyano, halogen, C₁₋₆ alkyl, C₁₋₆ alkoxy, C₃₋₆ cycloalkyl, C₁₋₆ haloalkyl, C₁₋₆ haloalkoxy, C₁₋₆ acylamino, or C₁₋₆ alkylamino; and
R₃, R₄, and R₅ are independently selected from hydrogen or C₁₋₆ alkyl

In an embodiment of the present disclosure, there is provided a compound of Formula (I), and its polymorphs, stereoisomers, prodrugs, solvates, co-crystals, intermediates, pharmaceutically acceptable salts, and metabolites thereof, wherein R is C₂ alkyl, amino acids, peptides, nucleobases, sugars, or lipids, wherein C₂ alkyl is optionally substituted with -NR₃R₄, -N⁺R₃R₄R₅, or -OC₁₋₆ alkyl, hydroxyl, cyano, C₁₋₆ alkoxy, C₁₋₆ haloalkyl, C₃₋₆ cycloalkyl, or C₅₋₁₀ aryl, wherein OC₁₋₆ alkyl, C₁₋₆ alkoxy, C₁₋₆ haloalkyl, C₃₋₆ cycloalkyl, or C₅₋₁₀ aryl is further substituted with hydroxyl, cyano, C₁₋₆ alkoxy, or C₁₋₆ haloalkyl;
R₁ is selected from hydrogen, -NR₃R₄, C₂₋₆ alkynyl, amino acids, peptides, nucleobases, sugars, or lipids, wherein C₂₋₆ alkynyl is substituted with 5-10 membered monocyclic aryl optionally substituted with 1-3 substituents selected from NR₃R₄, hydroxyl, cyano, halogen, C₁₋₆ alkyl, C₁₋₆ alkoxy, C₁₋₆ acylamino, or C₁₋₆ alkylamino; and
R₃, R₄, and R₅ are independently selected from hydrogen or C₁₋₆ alkyl

In an embodiment of the present disclosure, there is provided a compound of Formula (I), and its polymorphs, stereoisomers, prodrugs, solvates, co-crystals, intermediates, pharmaceutically acceptable salts, and metabolites thereof, wherein R is C₂ alkyl optionally substituted with -NH₂, -N(CH₃)₂, -⁺N(CH₃)₃, or -O-(CH₂)₂-OH;
R₁ is selected from hydrogen, NH₂, -N(CH₃)₂, or -C≡C-Ph, wherein -C≡C-Ph is optionally substituted with 1-3 substituents selected from NR₃R₄, hydroxyl, cyano, halogen, C₁₋₆ alkyl, C₁₋₆ alkoxy, C₁₋₆ acylamino, or C₁₋₆ alkylamino; and
R₃, R₄, R₅ are independently selected from hydrogen or C₁₋₆ alkyl.

In an embodiment of the present disclosure, there is provided a compound of Formula (I), and its polymorphs, stereoisomers, prodrugs, solvates, co-crystals, intermediates, pharmaceutically acceptable salts, and metabolites thereof, selected from:
a) 2-(6-((4-(dimethylamino)phenyl)ethynyl)-1,3-dioxo-1H-benzo[de]isoquinolin-2(3H)-yl)-N,N,N-trimethylethan-1-aminium;
b) 2-(2-aminoethyl)-6-((4-(dimethylamino)phenyl)ethynyl)-1H-benzo[de]isoquinoline-1,3(2H)-dione;
c) 6-((4-(dimethylamino)phenyl)ethynyl)-2-(2-(2-hydroxyethoxy)ethyl)-1H-benzo[de]isoquinoline-1,3(2H)-dione;
d) 2-(1,3-dioxo-1H-benzo[de]isoquinolin-2(3H)-yl)-N,N,N-trimethylethan-1-aminium;
e) 2-(6-(dimethylamino)-1,3-dioxo-1H-benzo[de]isoquinolin-2(3H)-yl)-N,N,N-trimethylethan-1-aminium, and
e) 2-(1,3-dioxo-6-(phenylethynyl)-1H-benzo[de]isoquinolin-2(3H)-yl)-N,N,N-trimethylethan-1-aminium.

In an embodiment of the present disclosure, there is provided a process of preparation of compound of Formula (I), and its polymorphs, stereoisomers, prodrugs, solvates, co-crystals, intermediates, pharmaceutically acceptable salts, and metabolites thereof, the process comprising reacting:

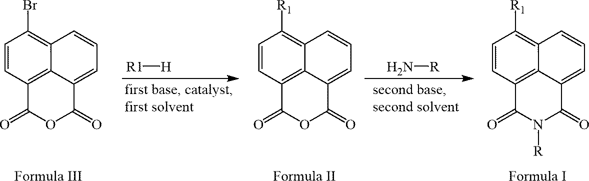

wherein R is selected from C₁₋₁₈ alkyl, C₂₋₁₈ alkenyl, C₂₋₁₈ alkynyl, C₅₋₁₀ aryl, C₁₋₁₈ heteroalkyl, C₅₋₁₈ heteroaryl, C₃₋₁₂ cycloalkyl, C₃₋₁₂ heterocyclyl, NR₃R₄, -N⁺R₃R₄R₅, -OC₁₋₁₈ alkyl, amino acids, peptides, nucleobases, sugars, or lipids, wherein C₁₋₁₈ alkyl, C₂₋₁₈ alkenyl, C₂₋₁₈ alkynyl, C₅₋₁₀ aryl, C₁₋₁₈ heteroalkyl, C₅₋₁₈ heteroaryl, C₃₋₁₂ cycloalkyl, C₃₋₁₂ heterocyclyl, -OC₁₋₁₈ alkyl is optionally substituted with one or more substituents selected from -NR₃R₄, -N⁺R₃R₄R₅, -OC₁₋₁₈ alkyl, hydroxyl, cyano, C₁₋₆ alkoxy, C₁₋₆ haloalkyl, C₃₋₆ cycloalkyl, C₅₋₁₀ aryl, C₃₋₆ heterocyclyl, or C₅₋₆ heteroaryl, wherein-OC₁₋₁₈ alkyl, C₁₋₆ alkoxy, C₁₋₆ haloalkyl, C₃₋₆ cycloalkyl, C₅₋₁₀ aryl, C₃₋₆ heterocyclyl, or C₅₋₆ heteroaryl is further substituted with hydroxyl, cyano, C₁₋₆ alkoxy, C₁₋₆ haloalkyl, C₃₋₆ cycloalkyl, C₅₋₁₀ aryl, C₃₋₆ heterocyclyl, or C₅₋₆ heteroaryl;
R₁ is selected from hydrogen, C₁₋₁₈ alkyl, C₂₋₁₈ alkenyl, C₂₋₁₈ alkynyl, C₅₋₁₀ aryl, C₁₋₁₈ heteroalkyl, C₅₋₁₈ heteroaryl, C₃₋₁₂ cycloalkyl, C₃₋₁₂ heterocyclyl, -NR₃R₄, -N⁺R₃R₄R₅, -OC₁₋₁₈ alkyl, amino acids, peptides, nucleobases, sugars, lipids, or -COOH, wherein C₁₋₁₈ alkyl, C₂₋₁₈ alkenyl, C₂₋₁₈ alkynyl, C₅₋₁₀ aryl, C₁₋₁₈ heteroalkyl, C₅₋₁₈ heteroaryl, C₃₋₁₂ cycloalkyl, C₃₋₁₂ heterocyclyl, OC₁₋₁₈ alkyl is optionally substituted with 5-10 membered monocyclic or bicyclic aryl optionally substituted with 1-5 substituents selected from NR₃R₄, hydroxyl, cyano, halogen, C₁₋₁₈ alkyl, C₁₋₁₈ alkoxy, C₃₋₁₂ cycloalkyl, C₁₋₁₈ haloalkyl, C₁₋₁₈ haloalkoxy, C₁₋₁₈ acylamino, or C₁₋₁₈ alkylamino; and R₃, R₄, and R₅ are independently selected from hydrogen or C₁₋₁₈ alkyl.

In an embodiment of the present disclosure, there is provided a process of preparation of compound of Formula (I) as disclosed herein, wherein the first base is selected from N,N-diisopropylethylamine (DIPEA), triethylamine (Et₃N), C₁₋₁₀ alkyl amine, or combinations thereof, the first solvent is selected dimethyl formamide, isopropyl alcohol, or combinations thereof; and the catalyst is selected from copper iodide, copper sulphate, sodium ascorbate, or combinations thereof.

In an embodiment of the present disclosure, there is provided a process of preparation of compound of Formula (I) as disclosed herein, wherein the second base is selected from N,N-diisopropylethylamine (DIPEA), triethylamine (Et₃N), C₁₋₁₀ alkyl amine, or combinations thereof; and the second solvent is selected from dimethyl formamide, isopropyl alcohol, or combinations thereof.

In an embodiment of the present disclosure, there is provided a process of preparation of compound of Formula (I) as disclosed herein, wherein reacting the compound of Formula II with R—NH₂ is carried out in microwave at a temperature in the range of 80 to 110° C. for a time period in the range of 4 to 7 hours. In another embodiment of the present disclosure, there is provided a process of preparation of compound of Formula (I) as disclosed herein, wherein reacting the compound of Formula II with R—NH₂ is carried out in microwave at a temperature of 0 100° C. for a time period of 6 hours.

In an embodiment of the present disclosure, there is provided a compound of Formula (I) as described herein for use in the manufacture of a medicament for treating a neurodegenerative disease.

In an embodiment of the present disclosure, there is provided a pharmaceutically acceptable salt of the compound of Formula (I) as described herein for use in the manufacture of a medicament for treating a neurodegenerative disease.

In an embodiment of the present disclosure, there is provided a compound of Formula (I) as described herein for use in the manufacture of a medicament for treating a neurodegenerative disease selected from amyotrophic lateral sclerosis (ALS), Alzheimer’s disease (AD), Parkinson’s disease (PD), prion diseases, polyglutamine expansion diseases, Huntington’s disease (HD), tauopathies, frontotemporal dementia associated with tau-immunoreactive inclusions (FTD-tau), progressive supranuclear palsy (PSP), or corticobasal degeneration (CBD).

In an embodiment of the present disclosure, there is provided a compound of Formula (I) as described herein, wherein the compound of Formula (I) modulates aggregation of Aβ42, tau, and α-syn.

In an embodiment of the present disclosure, there is provided a compound of Formula (I) as described herein, wherein the compound of Formula (I) provides reversal of cognitive decline or improvement of cognitive decline.

In an embodiment of the present disclosure, there is provided a pharmaceutically acceptable salt of the compound of Formula (I) as described herein, for use in the manufacture of a medicament for treating a neurodegenerative disease selected from amyotrophic lateral sclerosis (ALS), Alzheimer’s disease (AD), Parkinson’s disease (PD), prion diseases, polyglutamine expansion diseases, Huntington’s disease (HD), tauopathies, frontotemporal dementia associated with tau-immunoreactive inclusions (FTD-tau), progressive supranuclear palsy (PSP), or corticobasal degeneration (CBD).

In an embodiment of the present disclosure, there is provided a pharmaceutical composition comprising the compound of Formula (I) as described herein with a pharmaceutically acceptable carrier, optionally in combination with one or more other pharmaceutical compositions.

In an embodiment of the present disclosure, there is provided a pharmaceutical composition comprising a pharmaceutically acceptable salt of the compound of Formula (I) as described herein with a pharmaceutically acceptable carrier, in combination with one or more other pharmaceutical compositions. In another embodiment of the present disclosure, there is provided a pharmaceutical composition comprising a pharmaceutically acceptable salt of the compound of Formula (I) as described herein with a pharmaceutically acceptable carrier. In an embodiment of the present disclosure, there is provided a pharmaceutical composition as described herein, wherein the composition is in a form selected from tablet, capsule, powder, syrup, solution, aerosol, or suspension.

In an embodiment of the present disclosure, there is provided a compound of Formula (I) as described herein for the treatment of a condition mediated by a neurodegenerative disease, wherein an effective amount of the compound is administered to a subject in need thereof.

In an embodiment of the present disclosure, there is provided a pharmaceutical composition comprising the compound of Formula (I) as described herein for the treatment of a condition mediated by a neurodegenerative disease, wherein an effective amount of the composition is administered to a subject in need thereof.

In an embodiment of the present disclosure, there is provided a compound of Formula (I) as described herein for the treatment of a condition mediated by a neurodegenerative disease by administering a combination of the compound of Formula (I) with other clinically relevant immune modulator agents to a subject in need of thereof.

In an embodiment of the present disclosure, there is provided a pharmaceutical composition comprising the compound of Formula (I) as described herein for the treatment of a condition mediated by a neurodegenerative disease wherein administering a combination of the composition with other clinically relevant immune modulator agents to a subject in need of thereof.

In an embodiment of the present disclosure, there is provided a method for the treatment of a condition mediated by a neurodegenerative disease, said method comprising administering to a subject an effective amount of the compound of Formula (I) as described herein.

In an embodiment of the present disclosure, there is provided a method for the treatment of a condition mediated by a neurodegenerative disease, said method comprising administering to a subject an effective amount of the pharmaceutical composition as described herein.

In an embodiment of the present disclosure, there is provided a method of treatment of a condition mediated by a neurodegenerative disease, said method comprising administering a combination of the compound of Formula (I) as described herein with other clinically relevant immune modulator agents to a subject in need of thereof.

In an embodiment of the present disclosure, there is provided a method of treatment of a condition mediated by a neurodegenerative disease, said method comprising administering a combination of the pharmaceutical composition as described herein with other clinically relevant immune modulator agents to a subject in need of thereof.

In an embodiment of the present disclosure, there is provided a method for the treatment of a condition mediated by a neurodegenerative disease selected from amyotrophic lateral sclerosis (ALS), Alzheimer’s disease (AD), Parkinson’s disease (PD), prion diseases, polyglutamine expansion diseases, Huntington’s disease (HD), tauopathies, frontotemporal dementia associated with tau-immunoreactive inclusions (FTD-tau), progressive supranuclear palsy (PSP), or corticobasal degeneration (CBD).

In an embodiment of the present disclosure, there is provided a use of the compound of Formula (I) as described herein for treatment of a condition mediated by aggregation of Aβ42, tau, or α-syn.

In an embodiment of the present disclosure, there is provided a use of the pharmaceutical composition as described herein for the treatment of a condition mediated by aggregation of Aβ42, tau, or α-syn.

In an embodiment of the present disclosure, there is provided a use of the compound of Formula (I) as described herein with other clinically relevant agents or biological agents for the treatment of a condition mediated by aggregation of Aβ42, tau, or α-syn.

In an embodiment of the present disclosure, there is provided a use of the compound of the pharmaceutical composition as described herein with other clinically relevant agents or biological agents for the treatment of a condition mediated by aggregation of Aβ42, tau, or α-syn.

In an embodiment of the present disclosure, there is provided a compound of Formula (I) as described herein for use in treatment of a condition mediated by aggregation of Aβ42, tau, or α-syn.

In an embodiment of the present disclosure, there is provided a pharmaceutical composition comprising the compound of Formula (I) as described herein for use in treatment of a condition mediated by aggregation of Aβ42, tau, or α-syn.

In an embodiment of the present disclosure, there is provided a compound of Formula (I) as described herein with other clinically relevant agents or biological agents for treatment of a condition mediated by aggregation of Aβ42, tau, or α-syn.

In an embodiment of the present disclosure, there is provided a pharmaceutical composition comprising the compound of Formula (I) as described herein with other clinically relevant agents or biological agents for treatment of a condition mediated by aggregation of Aβ42, tau, or α-syn.

### EXAMPLES

The disclosure will now be illustrated with the working examples, which is intended to illustrate the working of disclosure and not intended to take restrictively to imply any limitations on the scope of the present disclosure. Unless defined otherwise, all technical and scientific terms used herein have the same meaning as commonly understood to one ordinary person skilled in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may apply.

### Materials and Methods

All reagents and solvents were procured from Sigma Aldrich or Specrochem without any further purification unless mentioned. Absorption and fluorescence spectra were recorded with Agilent Cary series UV-Vis-NIR absorption and Agilent Cary eclipse fluorescence spectrophotometers or SpectraMax i3x microplate reader (Molecular Devices), respectively. Data was plotted and analyzed in origin 8.5 or Prism 5. ¹H NMR and ¹³C NMR were performed using a Bruker AV-400 spectrometer with chemical shifts reported in parts per million (tetramethylsilane used as internal standard). Mass spectra were obtained from an Agilent 6538 UHD HRMS/Q-TOF high-resolution spectrometer. Aβ was purchased from Sigma Aldrich (USA). Polyoxyethylenesorbitan monolaurate (Tween 20) and skimmed milk were commercially procured from HIMEDIA laboratories. Anti-amyloid fibrils antibody (OC)was obtained from Merck biosciences (AB2286). RPMI media, fetal bovine serum (FBS), horse serum (HS), and pen-strep were obtained from Invitrogen. Fluorojade B was purchased from Milipore (USA). Anti-fade mounting medium with 4′,6-diamidino-2-phenylindole (DAPI) was procured from Vector Laboratories (Vectashield, Vector Laboratories, Burlingame, CA). The neuroblastoma (PC12) cells were commercially procured from GIBCO, Invitrogen and the neuronal cell rescue experiments were performed at Roswell Park Memorial Institute (RPMI) media. BioRad ECL kit (1705060) in Versa Doc instrument was obtained commercially from Bio-Rad laboratories.

### Example 1

### Synthesis of NMI Compounds

The compounds of Formula (I) were synthesized where R was C₂ alkyl optionally substituted with NH₂, —N(CH₃)₂, -⁺N(CH₃)₃, or —O—(CH₂)₂—OH and R₁ was selected from hydrogen, —N(CH₃)₂, or —C═C—Ph, wherein —C≡C—Ph was optionally substituted with —N(CH₃)₂. Pharmaceutically acceptable salts of the compounds may be obtained as per procedures reported in the literature. Scheme 1 represents the different compounds synthesized from the parent naphthalene monoanhydride molecule.

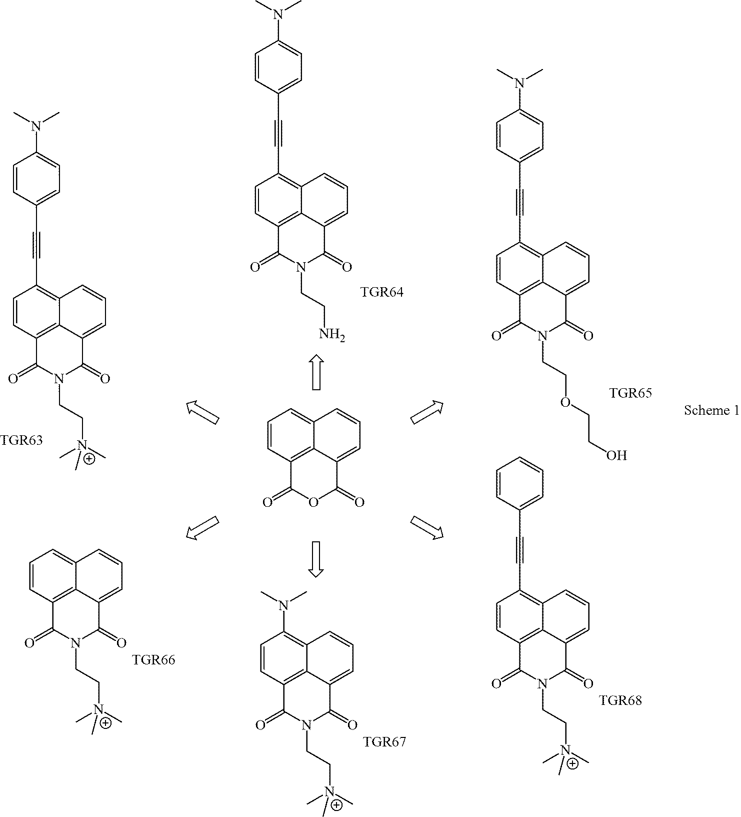

### Example 1.1: Synthesis of 4-((4-N,N Dimethylaniline)ethynyl)-1,8-naphthalic anhydride (Compound 1)

From 4-bromo-1,8-naphthalic anhydride, a formula of compound 1 was synthesized following Scheme 2. Compound 1 was further used as a reactant for the formation of TGR 63, TGR 64 and TGR 65. To a solution of 4-bromo-1,8-naphthalic anhydride (200 mg, 0.72 mmol) in a mixture of DMF/Et₃N (1 : 1) under argon, Pd(PPh₃)₄ (27 mg, 0.023 mmol), sodium ascorbate (10 mg, 50 µmol), copper (II) sulfate (2 mg, 8 µmol) and 4-ethynylanisole (93.6 µL, 0.72 mmol) were added. The reaction mixture was stirred for 4 h at 80° C. After completion of the reaction monitored by TLC, the reaction mixture was extracted with ethyl acetate, washed with NH₄Cl and brine, and dried over Na₂SO₄. The product was dissolved in ethyl acetate, precipitated with diethyl ether and collected by filtration. The compound was obtained as dark red coloured solid in 68% yield.

¹H NMR (CDCl₃, 400 MHz) δ 8.65 (d, 2H, J = 6.4), 8.64 (d, 2H, J = 4.2), 8.55 (d, 2H, J = 8), 7.92 (d, 2H, J = 7.6), 7.89 (t, 2H, J = 15), 7.55 (d, 2H, J = 4.2), 6.73 (d, 2H, J= 6.4), 3.06 (s, 6H); ¹³C NMR (CDCl₃, 100 MHz) δ 163.8, 151, 134.2, 133.6, 132.7, 131.5, 130.7, 130.4, 130, 127.4, 116.5, 111.7, 108, 40.1; HRMS (ESI-MS): found 342.1145, calcd. for C₂₂H₁₆NO₃ [M+H]⁺ m/z = 342.1112.

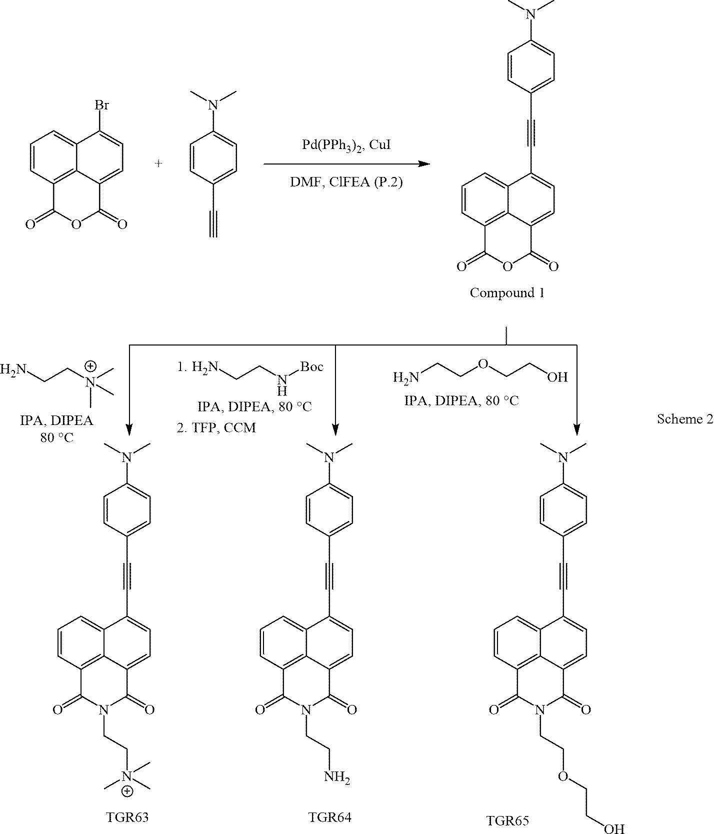

### Example 1.2: Synthesis of 2-(6-((4-(dimethylamino)phenyl)ethynyl)-1,3-dioxo-1H-benzo[de]isoquinolin-2(3H)-yl)-N,N,N-trimethylethan-1-aminium (TGR 63)

To a solution of compound 1 (200 mg, 0.58 mmol) dispersed in isopropanol (IPA), a mixture of DIPEA (31 mL, 1.7 mmol) and 2-amino-N,N,N-trimethylethanaminium (60 mg, 0.58 mmol) was added and refluxed at 80° C. for 6 h. The reaction mixture was extracted with ethyl acetate, washed with brine, and dried over Na₂SO₄. The crude product was purified using column chromatography on silica gel using 1% MeOH in CHCl₃ as an eluent to afford a red colored solid in good yield (75%). ¹H NMR (DMSO d₆, 400 MHz) δ 8.60 (d, 1H, J = 0.8), 8.58 (d, 1H, J = 1.2), 8.48 (d, 1H, J = 7.6), 8.03 (d, 2H, J = 4.4), 8.01 (t, 2H, J = 4.8), 7.61 (d, 2H, J = 2), 6.80 (d, 2H, J = 8.8), 4.48 (t, 2H, J = 13.6), 3.65 (t, 2H, J = 14.8), 3.21 (s, 9H), 3.01 (s, 6H); ¹³C NMR (DMSO d₆, 100 MHz) δ 163.3, 163, 158, 150.9, 133.2, 132.5, 131.3, 130.6, 130.4, 129.7, 128, 127.6, 122.4, 120.5, 111.8, 106.9, 102.3, 85.9, 52.4, 33.6; HRMS (ESI-MS): found 426.2176, calcd. for C₂₇H₂₈N₃O₂ [M]⁺ m/z = 426.2176.

### Example1.3: Synthesis of 2-(2-aminoethyl)-6-((4-(dimethylamino)phenyl)ethynyl)-1H-benzo[de]isoquinoline-1,3(2H)-dione (TGR 64)

To a solution of compound 1 (200 mg, 0.58 mmol) dispersed in isopropanol, a mixture of DIPEA (31 µL, 1.7 mmol) and tert-butyl 2-aminoethylcarbamate (39 mg, 0.58 mmol) was added and refluxed at 80° C. for 6 h. The reaction mixture was extracted with ethyl acetate, washed with brine, and dried over Na₂SO₄. The crude product was purified using column chromatography on silica gel using 0.25% MeOH in CHCl₃ as an eluent to afford a red colored solid. Then the compound was deprotected using TFA (95% TFA, 4.5% DCM and 0.5% TIPS) and precipitated to obtain pure product in good yield (68%). ¹H NMR (DMSO d₆, 400 MHz) δ 8.77 (d, 1H, J = 1.8), 8.56 (d, 1H, J = 3.6), 8.45 (d, 1H, J = 3.8), 8.00 (d, 2H, J = 3), 7.97 (d, 2H, J = 8.8), 7.59 (d, 2H, J = 3.3), 6.74 (d, 2H, J = 3.8), 4.33 (t, 2H, J = 11.6), 3.17 (s, 2H), 3.01 (s, 6H); ¹³C NMR (DMSO d₆, 100 MHz) δ 163.8, 163.5, 150.8, 133.2, 132.2, 131.1, 130.5, 130.1, 129.7, 127.9, 127.7, 122.7, 120.8, 111.8, 107, 102, 85, 37.6, 37.5; HRMS (ESI-MS): found 383.1767, calcd. for C₂₄H₂₁N₃O₂ [M]⁺ m/z = 383.1634.

### Example 1.4: Synthesis of 6-((4-(dimethylamino)phenyl)ethynyl)-2-(2-(2-hydroxyethoxy)ethyl)-1H-benzo[de]isoquinoline-1,3(2H)-dione, (TGR 65)

To a solution of compound 1 (200 mg, 0.58 mmol) dispersed in isopropanol, a mixture of DIPEA (31 µL, 1.7 mmol) and 2-(2-aminoethoxy)ethanol (22 mL, 0.58 mmol) was added and refluxed at 80° C. for 6 h. The reaction mixture was extracted with ethyl acetate, washed with brine, and dried over Na₂SO₄. The crude product was purified using column chromatography on silica gel with CHCl₃ as an eluent to afford a red colored solid in good yield (72%). ¹H NMR (DMSO d₆, 400 MHz) δ 8.76 (d, 1H, J= 8.4), 8.55 (d, 1H, J = 7.2), 8.43 (d, 1H, J = 7.6), 7.98 (d, 2H, J = 8), 7.95 (t, 2H, J = 1.6), 7.59 (d, 2H, J = 8.8), 6.79 (d, 2H, J = 8.8), 4.25 (t, 2H, J = 12.8), 3.67 (t, 2H, J= 12.8), 3.47 (s, 4H), 3.31 (s, 4H), 3.00 (s, 6H); ¹³C NMR (DMSO d₆, 100 MHz) 163.2, 162.9, 133.2, 132.1, 131.1, 130.5, 130.2, 129.7, 127.9, 127.6, 127.5, 122.4, 120.6, 111.8, 107, 101.8, 85, 72, 66.8, 60.1, 28.9; HRMS (ESI-MS): found 429.1803, calcd. for C₂₆H₂₅N₂O₄ [M+H]⁺ m/z = 429.1814

### Example 1.5: Synthesis of 2-(1,3-dioxo-1H-benzo[de]isoquinolin-2(3H)-yl)-N,N,N-trimethylethan-1-aminium (TGR 66)

To a solution of 1,8-naphthalic anhydride (Compound 2, 114 mg, 0.58 mmol) dispersed in isopropanol, a mixture of DIPEA (31 µL, 1.7 mmol) and 2-amino-N,N,N-trimethylethanaminium (60 mg, 0.58 mmol) was added and refluxed at 80° C. for 6 h. The reaction mixture was extracted with CHCl₃, washed with brine, and dried over Na₂SO₄ (Scheme 3). The crude product was purified using column chromatography on silica gel using 2% MeOH in CHCl₃ as an eluent to afford a white solid in good yield (88%) (Scheme 3). ¹H NMR (DMSO d₆, 400 MHz) δ 8.54-8.50 (m, 4H), 7.93-7.89 (m, 2H), 4.49 (t, 2H, J = 14.4), 3.66 (t, 2H, J = 14.4), 3.23 (s, 9H); ¹³C NMR (DMSO d₆, 100 MHz) δ 163.4, 134.7, 131.3, 130.9, 127.4, 127.3, 121, 89, 61.9, 52.5, 33.6; HRMS (ESI-MS): found 283.1439, calcd. for C₁₇H₁₉N₂O₂ [M]⁺ m/z = 283.1441

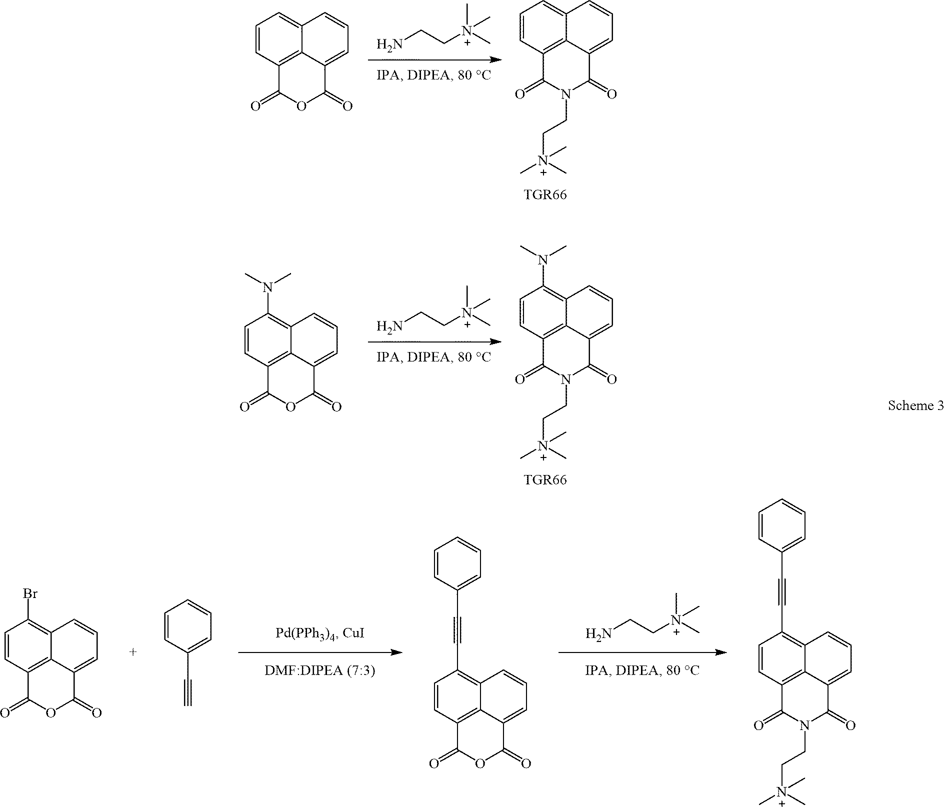

### Example 1.6: Synthesis of 2-(6-(dimethylamino)-1,3-dioxo-1H-benzo[de]isoquinolin-2(3H)-yl)-N,N,N-trimethylethan-1-aminium (TGR 67)

To a solution of 4-dimethylamine-1,8-naphthalic anhydride (139 mg, 0.58 mmol) dispersed in isopropanol, a mixture of DIPEA (31 µL, 1.7 mmol) and 2-amino-N,N, -trimethylethanaminium (60 mg, 0.58 mmol) was added and refluxed at 80° C. for 6 h. the reaction mixture was extracted with CHCl₃, washed with brine, and dried over Na₂SO₄ (Scheme 3). The crude product was purified using column chromatography on silica gel using 3.5% MeOH in CHCl₃ as an eluent to afford a yellow solid in appropriate yield (54%). ¹H NMR (DMSO d₆, 400 MHz) δ 8.50 (d, 1H, J= 6.4), 8.49 (d, 1H, J = 4.2), 8.38 (d, 1H, J= 8.4), 7.80 (d, 1H, J= 7.2), 7.78 (d, 1H, J= 7.2), 7.24 (d, 1H, J = 4.2), 4.96 (t, 2H, J = 14), 3.64 (t, 2H, J = 14), 3.20 (s, 9H), 3.12 (s, 6H); ¹³C NMR (DMSO d₆, 100 MHz) 163.7, 162.9, 156.9, 132.6, 132.1, 130.8, 124.9, 124, 122, 112.8, 112.5, 52.4, 44.3, 33.4; HRMS (ESI-MS): found 326.1864, calcd. for C₁₉H₂₄N₃O₂ [M]⁺ m/z = 326.1863.

### Example 1.7: Synthesis of 2-(1,3-dioxo-6-(phenylethynyl)-1H-benzo[de]isoquinolin-2(3H)-yl)-N,N,N-trimethylethan-1-aminium (TGR 68)

To a solution of 4-(benzylethynyl)-1,8-naphthalic anhydride (172 mg, 0.58 mmol) dispersed in isopropanol, a mixture of DIPEA (31 µL, 1.7 mmol) and 2-amino-N,N,N-trimethylethanaminium (60 mg, 0.58 mmol) was added and refluxed for 6 h. The reaction mixture was extracted with CHCl₃, washed with brine, and dried over Na₂SO₄ (Scheme 3). The crude product was purified using column chromatography on silica gel using in CHCl₃ as an eluent to afford a yellow solid in good yield (74%). ¹H NMR (DMSO d₆, 400 MHz) δ 8.83 (d, 1H, J= 8.4), 8.61 (d, 1H, J= 7.2), 8.51 (d, 1H, J= 7.6), 8.13 (d, 1H, J= 7.6), 8.05 (t, 1H, J = 15.6), 7.81-7.78 (m, 2H), 7.54-7.52 (m, 3H), 4.49 (t, 2H, J = 14.4), 3.67 (t, 2H, J = 14.4), 3.24 (s, 9H); ¹³C NMR (DMSO d₆, 100 MHz) 163.2, 162.9, 132.3, 131.9, 131.4, 131, 130.9, 130.2, 129.9, 128.9, 128.4, 127.4,5, 126.5, 122.5, 121.8, 121.2, 99, 86.0, 61.9, 54.8, 52.4, 33.7; HRMS (ESI-MS): found 384.1693, calcd. for C₂₅H₂₃N₂O₂ [M]⁺ m/z = 384.1854.

### Example 1.8 Synthesis of Compound of Formula (I) With Unnatural Amino Acid

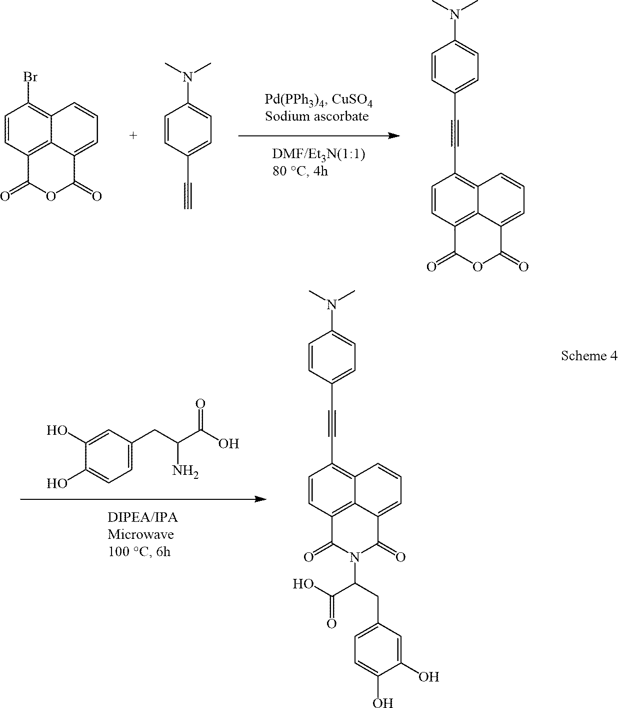

Scheme 4 depicts the synthesis of compound of Formula (I) with unnatural amino acid. To a stirred solution of 4-Bromo-1,8-naphthalic anhydride (200 mg, 0.72 mmol) was dissolved in DMF and triethylamine (3 mL, 1:1) and 4-ethynyl-N,N-dimethylaniline (105 mg, 0.72 mmol) and Pd(PPh₃)₄ (42 mg, 0.03 mmol) were added. The reaction mixture was allowed to stir for 10 min and sodium ascorbate (7 mg, 0.03 mmol) and CuSO₄ (2.3 mg, 0.01 mmol) were added. Then the resulting mixture was heated at 80° C. for 4 hours and the progression of the reaction was monitored through TLC. After the reaction was completed, the solvent was evaporated, and the crude product was dissolved in EtOAc and washed with NH₄Cl solution, water, and brine solution and dried over anhydrous Na₂SO₄. The product was purified under column chromatography using EtOAc-Hexane as a mobile phase to get pure intermediate at good yield (~61%). Next, anhydride intermediate (20 mg, 0.06 mmol) and L-DOPA (11.5 mg, 0.06 mmol) were dissolved in isopropanol and DIPEA (0.1 mL) was added to the mixture and heated in the microwave at 100° C. for 6 hours (3 × 2 h). After the reaction was completed, the solvent was evaporated. The residue was dissolved in EtOAc and cold HCl (0.2 M) was added and the solvent mixture was stirred for 30 min. Finally, the precipitate was filtered and washed thoroughly with DCM to get pure product. ¹H-NMR (400 MHz, DMSO-D₆) δ 8.80 (d, J=8 Hz, 1H), 8.54 (d, J=8 Hz, 1H), 8.42 (d, J=8 Hz, 1H), 7.99 (t, J=4 Hz, 2H), 7.61 (d, J=12 Hz, 2H), 6.80 (d, J=8 Hz, 2H), 6.54 (s, 1H), 6.44 (d, J=8 Hz, 1H), 6.36 (d, J-8 Hz, 1H), 5.79 (dd, J=8, 4 Hz, 1H), 3.40 (dd, J=8, 4 Hz, 1H), 3.24 (dd, J=12, 4 Hz, 1H), 3.01 (s, 6H). ¹³C-NMR (100 MHz, DMSO-D6) δ 170.6, 162.7, 162.4, 150.8, 144.6, 163.4, 133.2, 132.5, 131.6, 130.6, 130.4, 129.7, 128.4, 128.0, 127.3, 121.7, 119.7, 119.5, 116.2, 115.2, 111.7, 106.9, 102.2, 85.0, 54.1, 33.4. HRMS (ESI-MS) : found 521.1690, calcd. For C₃₁H₂₄N₂O₆ [M+H]⁺ m/z=521.1634.

### Example 1.9 Synthesis of Compound of Formula (I) With Natural Amino Acid

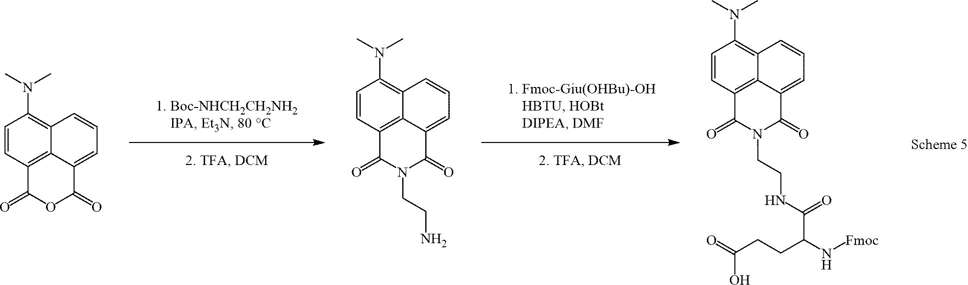

Scheme 5 depicts the synthesis of compound of Formula (I) with natural amino acid. To a stirred solution of 4-dimethylamine-1,8-naphthalic anhydride (200 mg, 0.83 mmol) was dissolved in isopropanol (IPA, 15 mL) and Boc-protected ethylene diamine (172.5 mg, 1.1 mmol) and triethylamine (0.23 mL, 1.66 mmol) were added. The reaction mixture was refluxed for 12 h under the nitrogen atmosphere. After the completion of the reaction (monitored by TLC), the excess solvent was removed, the crude was diluted with water (20 mL), and the residue was extracted into EtOAc (3 × 20 mL). The combined organic phase (EtOAc) was washed with water (1 × 25 mL) and brine (1 × 30 mL). The organic layers were combined and dried on anhydrous Na₂SO₄ and evaporated. The product was purified by column chromatography using EtOAc and hexane as eluent. Next, the intermediate (0.5 g, 1.5 mmole) was dissolved in DCM (5 mL) and TFA (2 mL) was added, and the reaction mixture was stirred for 3h at room temperature. The solvent was removed, and the product was precipitated with cold diethyl ether (25 mL). Next, to a stirred solution of intermediate (215 mg, 0.51 mmol) in DMF (10 mL) at 0° C., DIPEA (0.17 mL, 1.02 mmol), HBTU (232.1 mg, 0.61 mmol), and HOBt (83.0 mg, 0.61 mmol) were added. The reaction mixture was kept for stirring about 20 min under a nitrogen atmosphere, and Fmoc-Glu(OtBu)-OH (2.15 mg, 0.51 mmol) was added to the solution; the reaction was left to stir for 5-6 h at room temperature. After completing the reaction (monitored by TLC), the DMF was removed. The crude was diluted with water (25 mL), and the residue was extracted into EtOAc (3 ×20 mL). The combined organic layer (EtOAc) was washed with water (1 × 25 mL) and brine (1 ×25 mL), dried over anhydrous Na₂SO₄ and evaporated under vacuum to afford the crude peptide. The intermediate was purified by column chromatography using DCM and methanol as eluent. Finally, intermediate (0.3 g, 0.43 mmole) was dissolved in DCM (10 mL), and TFA (2 mL) was added, and the reaction mixture was stirred for 3 h at room temperature. The DCM was removed under vacuum, and the crude NGlu was precipitated in cold diethyl ether. ¹H-NMR (600 MHz, DMSO-D6) δ 12.07 (s, 1H), 8.42-8.48 (m, 2H), 8.30 (d, J = 8.5 Hz, 1H), 8.03 (t, J = 5.9 Hz, 1H), 7.87 (d, J = 7.5 Hz, 2H), 7.71 (dd, J = 13.6, 7.6 Hz, 3H), 7.40 (t, J = 8.2 Hz, 3H), 7.30 (dd, J = 11.9, 7.3 Hz, 2H), 7.16 (d, J = 8.3 Hz, 1H), 4.10-4.22 (m, 5H), 3.85 (td, J = 8.4, 5.2 Hz, 1H), 3.29 (dd, J = 13.2, 6.0 Hz, 1H), 3.05 (s, 6H), 2.19 (dd, J = 15.4, 9.0 Hz, 2H), 1.83 (td, J = 14.4, 5.9 Hz, 1H), 1.59-1.66 (m, 1H), 1.14-1.32 (m, 1H). ¹³C-NMR (150 MHz, DMSO-D6) δ 173.9, 171.4, 163.8, 163.2, 156.4, 155.8, 143.8, 143.7, 140.6, 132.2, 131.2, 130.5, 129.7, 127.6, 127.0, 125.3, 125.3, 124.9, 124.2, 122.5, 120.0, 115.6, 113.6, 112.9, 79.1, 78.9, 78.7, 65.6, 54.0, 46.6, 44.3, 36.7, 34.3, 30.2, 28.9, 26.9. HRMS (ESI-MS): found 635.2525, calcd. For C₃₆H₃₄N₄O₇ [M+H]⁺ m/z= 635.2506.

### Example 1.10 Synthesis of Compound of Formula (I) With Peptide

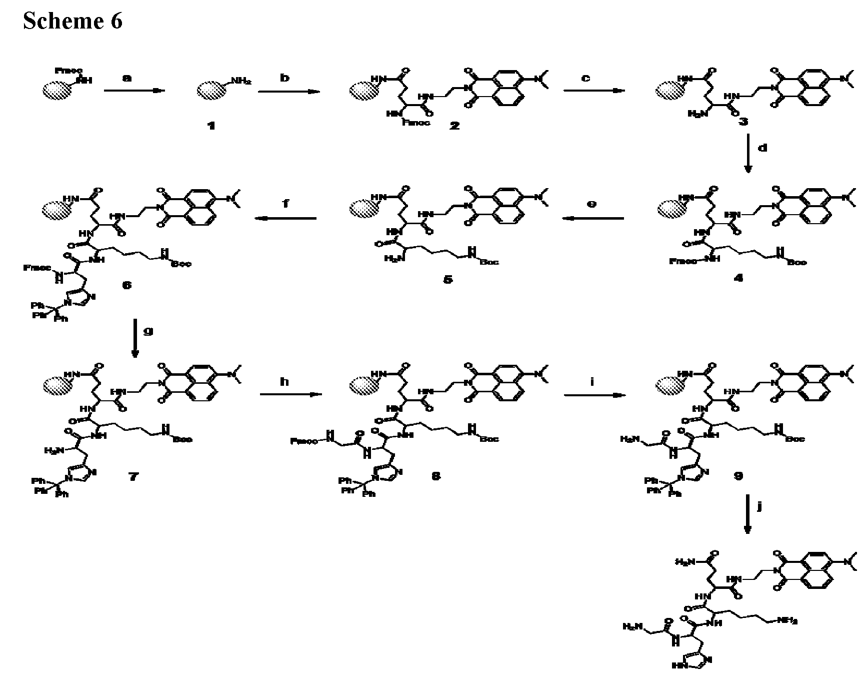

a: DCM, DMF, Pip (20%); b: NTP, HBTU, HOBt, DIPEA, DMF; c: DMF, Pip (20%); d: Fmoc-Lys(Boc)-OH, HBTU, HOBt, DIPEA, DMF; e: DMF, Pip (20%); f: Fmoc-His(trt)-OH, HBTU, HOBt, DIPEA, DMF; g: DMF, Pip (20%); h: Fmoc-Gly-OH, HBTU, HOBt, DIPEA, DMF; i: DMF, Pip (20%); and j: DCM, TFA.

Rink amide resin (100 mg) was washed with DCM (3 × 3 mL) and DMF (3 × 3 mL), and the resin was kept in DCM (3 mL) for 30 min under shaking conditions. The DCM was removed, and again DMF (5 mL) containing piperidine (Pip, 20 %) was added; the reaction mixture was subjected to vigorous shaking for 30 min at room temperature. The DMF was removed, and the resin was washed with DCM (3 × 3 mL) and DMF (3 × 3 mL). The 1 was dispersed in DMF (4 mL), and 1,8-naphthaline imide-attached amino acid (98.5 mg, 2 equivalent), DIPEA (0.8 mL, 4 equivalent), HBTU (118.0 mg, 4 equivalent), and HOBt (42.0 mg, 4 equivalent) were added, and the reaction mixture has been subjected to vigorous shaking for 4 h at room temperature (Scheme 6). The completion of the reaction was monitored by the Kaiser test. After the completion of the reaction, the solvent was removed, and DMF (5 mL) containing Pip (20 %) was added to intermediate 2, and allowed to react for 30 min at room temperature. Next, 3 was dispersed in DMF (5 mL) and Fmoc-Lys(Boc)-OH (146.2 mg, 4 equivalent), DIPEA (0.8 mL, 4 equivalent), HBTU (118.0 mg, 4 equivalent), and HOBt (42.0 mg, 4 equivalent) were added; and the reaction mixture was again subjected to vigorous shaking for 2 h at room temperature. After the completion of the reaction, the solvent was removed, and DMF (5 mL) containing Pip (20 %) was added in intermediate 4, and allowed to react for 30 min at room temperature. Next, 5 was dispersed in DMF (5 mL) and Fmoc-His(trt)-OH (193.4 mg, 4 equivalent), DIPEA (0.8 mL, 4 equivalent), HBTU (118.0 mg, 4 equivalent), and HOBt (42.0 mg, 4 equivalent) were added; and the coupling reaction was carried out for 5 h at room temperature. After the completion of the reaction, the solvent was removed, and DMF (5 mL) containing Pip (20 %) was added in intermediate 6 to obtain intermediate 7. Next, 7 was dispersed in DMF (5 mL) and Fmoc-Gly-OH (92.7 mg, 4 equivalent), DIPEA (0.8 mL, 4 equivalent), HBTU (118.0 mg, 4 equivalent), and HOBt (42.0 mg, 4 equivalent) were added; and the reaction mixture again allowed to vigorous shaking for 3 h at room temperature. After the completion of the reaction, the solvent was removed, and DMF (5 mL) containing Pip (20 %) was added in intermediate 8 to obtain intermediate 9. Finally, the 9 was dispersed in DCM (3 mL), and TFA (1 mL) was added to the solution to obtain crude peptide, which was purified using a reverse-phase (RP) semipreparative HPLC on the C18 column at 40° C. and the integrity of the product was ascertained by analytical liquid chromatography-mass spectrometry (LCMS) analysis.

### Example 2

### Preparation of Aβ42 Monomeric and Fibrillar Samples

Monomeric form of Aβ42 peptide was prepared by dissolving 0.25 mg of commercially available β-amyloid peptide (1-42), human (cat: PP69-.025 mg), marck peptide in 250 µL of hexafluoro-2-propanol (HFIP) solvent to obtain a Aβ42 solution. This solution was incubated at room temperature for 1 hour, further to which the HFIP solvent was removed by nitrogen gas flow to obtain the monomeric Aβ42 peptide. To prepare a fibrillar sample of Aβ42 peptide, 0.025 mg of Aβ42 peptide was dissolved in 55.6 mL of PBS buffer (pH = 7.4; contains 2% of DMSO or 100 mM NaOH) to obtain a monomeric Aβ42 solution and the peptide concentration in this solution was calculated through UV-Visible absorbance studies (ε = 1450 cm⁻¹ M⁻¹). The Aβ42 monomeric solution was incubated for 5 days in PBS buffer (pH = 7.4) to allow for the full phase formation of Aβ42 fibrils. The presence of Aβ42 fibrils was further confirmed by ThT assay.

### Example 3

### Inhibition and Dissolution of Fibrillar Assembly

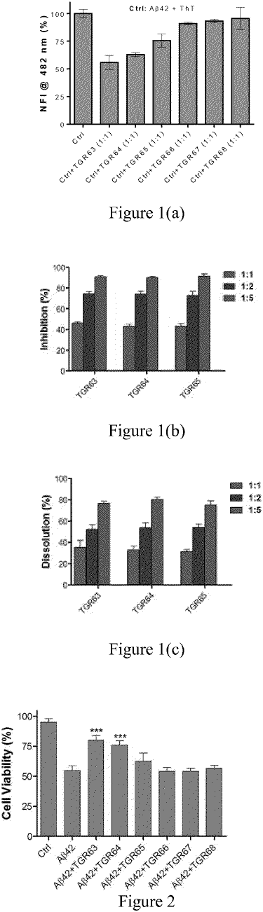

Initially, the ability of the prepared NMI compounds, TGR63-68 to inhibit Aβ42 fibrillar assembly at 1:1 molar ratio with Aβ42 fibrils was evaluated using thioflavin (ThT) binding assay. 10 µM of Aβ42 fibrils in the absence (Ctrl) and the presence of 10 µM of inhibitors individually were incubated for 72 h. A normalized data for fluorescence intensity (NFI) of ThT at 482 nm (λex = 442 nm) was measured as represented in FIG. 1(a). It was observed that TGR63-65 showed a significant inhibitory effect on Aβ42 aggregation, ~45%, 38% and 25%, respectively. While on the other hand, TGR66-68 showed minimal effects on Aβ42 aggregation, ~10%, 7% and 5%, respectively. Overall, TGR63 was found to be the most effective inhibitor among others (TGR64-68). Since, TGR 66-68 did not show appreciable performance with respect to inhibition of Aβ42 fibrils, they were exempted from following studies conducted at varying concentration ratios.

The ability of NMI compounds (TGR63, TGR64 and TGR65) to prevent Aβ42 fibrillar assembly (inhibition) and to break down the preformed fibrils (dissolution) was evaluated using thioflavin (ThT) binding assay. Aβ42 fibrils at a concentration of 10 µM and its aggregates, were used to study the effect of NMI compounds (TGR63, TGR64 and TGR65) at varying concentrations on both inhibition and dissolution assays. For the inhibition assay, all the NMI compounds (TGR63, TGR64 and TGR65) were added independently to Aβ peptide (10 µM) at 0 h of the experiment, whereas for the aggregates dissolution assay they were added to Aβ42 fibrillar aggregates grown for 2 days. Upon incubating for a predetermined period, Aβ42 inhibitor samples were analyzed using ThT by measuring the fluorescence change at 485 nm as presented in FIG. 1(b) and FIG. 1(c), wherein the error bars represent the standard deviation (SD) of the fluorescence measurement. Fluorescence values were normalized to maximal fluorescence intensity at 485 nm compared to that of the control (Aβ42 with no inhibitor). Experiments were performed at various stoichiometric ratios (Aβ42/inhibitor) of 1:1, 1:2, and 1:5 with a fixed concentration of Aβ42 at 10 µM. Each experiment was repeated three times (n = 3). Results for inhibition observed at third day of incubation, as recorded in FIG. 1(b) demonstrated that TGR63, TGR64 and TGR65 were able to prevent Aβ42 aggregation in a concentration-dependent inhibition fashion. At 1:1 ratio, all the inhibitors (TGR63, TGR64 and TGR65) showed ~50 % decrease in the formation of Aβ aggregates. Further, an increase in the concentration of inhibitors to ratios 1:2 and 1:5, effectively decreased the Aβ aggregation to 75% and 90%, respectively. Similar results were observed in the case of fibril reversal assay with dissolution efficiencies measured after the sixth day of incubation as depicted in FIG. 1(c). TGR 63 recorded 80% dissolution efficiency while TGR 64 and TGR 65 recorded 75% and 78% dissolution efficiency respectively, for the stoichiometric ratio of 1:5. At 1:1 and 1:2 ratios, inhibitors TGR63, TGR64, and TGR65 showed dissolution efficiencies of ~35% and ~50%, respectively. Thus, TGR63, TGR64 and TGR65 were found to be promising molecules as they displayed a pronounced effect in both inhibition and dissolution assays.

### Example 4

### Rescue of Neuronal Cells From Amyloid Toxicity

To demonstrate the neuronal cell rescue ability of the prepared NMI compounds (TGR63-68) from Aβ42 peptide toxicity, cells rescue assay was performed with neuroblastoma (PC12) cells. A 96-well plate was used to culture the cells (15,000 per well) with RPMI media (10% fetal bovine serum (FBS), 5% horse serum (HS,), and 1% pen-strep (PS)) at 37° C. temperature in a 5% CO₂ atmosphere. Then the media was exchanged with low serum (2% FBS) media and monomeric Aβ42 was added in the absence and presence of NMI compounds and incubated for 24 h. The efficiency of TGR63, TGR64 and TGR65 inhibitors in modulating Aβ aggregates induced cellular toxicity was analysed. For this purpose, the ability of TGR63, TGR64 and TGR65 to rescue PC12 cells from Aβ42 toxicity was studied through cell viability assay (MTT assay). Cellular viability was observed after incubation of 24 h with Aβ42 (20 µM) alone and in combination with TGR63 (40 µM), TGR64 (40 µM), and TGR65 (40 µM), independently. Each experiment was repeated three times (n = 3), the results for which are revealed in FIG. 2, wherein the error bars represent the standard deviation (SD). FIG. 2 shows that TGR65 exhibited only a slight improvement in cell viability (62%) as compared to Aβ42 treated cells (54%). Remarkably TGR63 and TGR64 showed improved cell viability to 80% and 78%, respectively. ThT binding assay showed that NMI compounds (TGR63, TGR64 and TGR65) exhibited similar amyloid aggregation inhibition property, and a similar trend in the rescue of PC12 cells from amyloid toxicity was observed. However, TGR63 and TGR64 exhibited enhanced protection, indicating that improved cell viability is not solely driven by aggregation modulation. Therefore, TGR63 and TGR64 would possibly be involved in other cellular mechanisms to exhibit better cellular viability from inhibition of amyloid toxicity.

### Example 5

### Structure-Activity Relationship

The structure-activity relationship (SAR) of NMI compounds towards rescuing PC12 cells from Aβ toxicity was investigated. The lead derivatives TGR63 and TGR64 were kept structurally similar except for the quaternary amine in TGR63, and a primary amine in TGR64. FIG. 2 illustrates cell viability observed after incubation of 24 h with Aβ42 (20 µM) alone and in combination with TGR63 (40 µM), TGR64 (40 µM), TGR65 (40 µM), TGR66 (40 µM), TGR67 (40 µM), and TGR68 (40 µM), independently. Each experiment was repeated three times (n = 3), and error bars represent the standard deviation (SD). The absence of amine in the TGR65 showed only a slight improvement in the cell viability from Aβ toxicity which indicated that ethylenediamine has a significant role in rescuing the cells. TGR63 showed slightly better cellular rescue from Aβ toxicity compared to TGR64, which indicated that N,N,N-trimethylethylenediamine is an essential structural moiety. Thus, this moiety was retained in designing further analogues for structure-activity study. To evaluate the role of 4-ethynyl-N,N-dimethylbenzenamine (EMB) moiety present in TGR63 and TGR64, TGR66 was synthesized with the absence of EMB. TGR66 showed cell viability of only 54% indicating that it could not rescue the cells from Aβ toxicity which further indicated that EMB in TGR63 and TGR64 play a significant role in enhancing the cell viability. TGR67, structurally similar to TGR63, with N,N-dimethylamine group directly coupled to 8th position of NMI, and TGR68 without EMB group and ethynylbenzene group directly coupled to 8th position of NMI, were synthesized. In cellular viability assay, TGR67 and TGR68 showed cell viability of 54% and 56% respectively. Thus, it can be inferred that structural changes in EMB present in TGR63-65 substantially reduced the protective nature of NMI compounds in TGR66-68, indicating that EMB is an essential structural moiety for the rescue of cells from Aβ toxicity. Moreover, from these studies, TGR63 was found to be the most potent compound, and therefore all further studies were performed using TGR63.

### Example 6

### Molecular Interactions Among Aβ42 Peptides and TGR63

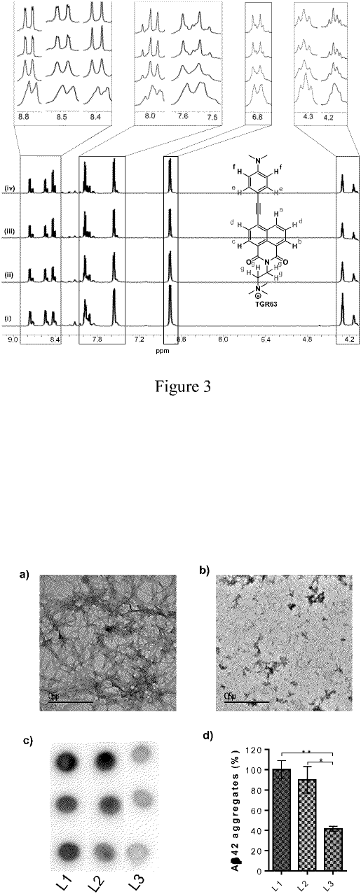

Nuclear magnetic resonance (NMR) spectroscopy was performed to ascertain the molecular level interactions between TGR63 and Aβ42 peptide. ¹H NMR spectra of TGR63 were acquired in absence and presence of Aβ42 (10 µM) peptides using the WATERGATE sequence for solvent suppression in deuterium oxide (D₂O, 12%) containing PBS at different incubation time points (24, 48 and 72 h). The ¹H NMR spectra of only TGR63 is displayed in FIG. 3. The aromatic protons of NMI and aniline moieties (a-f) appeared at 6.5-8.8 ppm, respectively. However, the splitting patterns of all the aromatic peaks were completely readjusted undergoing a significant downfield shift over a period of time in the presence of Aβ42 peptide. This observation confirmed the interaction of aromatic moieties (π-electron rich) of TGR63 with native and misfolded Aβ42 peptides, which leads to alteration in the extent of nuclei and proximal electron coupling (J-couplings). The downfield shift and change in J-couplings of ethyl hydrogen (g) at 4.1-4.4 ppm clearly indicated their interaction with Aβ42 peptides and revealed their involvement during amyloidogenesis. Overall, the solution phase ¹H NMR study demonstrated that TGR63 interacts with native and misfolded Aβ42 peptides and modulate the toxic β-sheet formation by interfering with the essential noncovalent interactions.

### Example 7

### Visualization of Aβ42 Inhibition Using TGR63

The importance of the involvement of TGR63 during amyloidogenesis was evaluated through amyloid fibrillar structure analysis using transmission electron microscopy (TEM) and dot blot analysis.

### Transmission Electron Microscopy

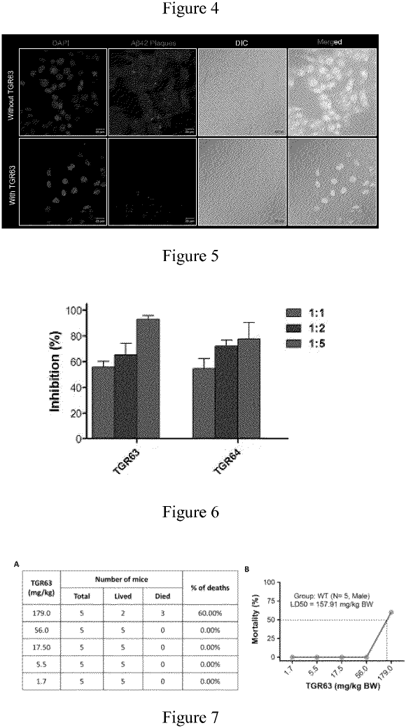

To visualize the amyloid fibrils, the 10 µM of Aβ42 peptides were incubated alone, and in combination with 50 µM TGR63 for 48 h and spotted on mica surface and TEM grid to acquire the TEM images. FIG. 4(a) and FIG. 4(b) show the TEM images of Aβ42 aggregation species in the absence and presence of TGR63 respectively. The obtained TEM images in FIG. 4(a) were found to be in very good agreement with the ThT results obtained in Example 2 and displayed a highly intertwined structure of Aβ42 fibrils which was then observed to be disrupted in presence of TGR63 in FIG. 4(b). Thus, the visualisation of aggregated amyloid species in the absence and presence of TGR63 clearly displayed the amyloidogenesis inhibition and reduction in amyloid fibrils formation.

### Dot-Blot Analysis

The inhibition ability of TGR63 was further supported through dot blot (immunohistochemistry) analysis which was used to evaluate the amount of Aβ42 fibrils. 10 µM of freshly prepared Aβ42 sample was incubated with TGR63 and alone independently for 48 h without shaking. The incubated samples were dotted on the PVDF membrane and allowed to dry. The PVDF membranes were blocked using 5% skimmed milk in PBS for 1 h at room temperature. The blots were washed (3 times) with 1% of Tween 20 containing PBS (PBST) for 10 min and incubated with OC (1:1000) primary antibody, specific to Aβ42 fibrils at 4° C. for 16 h. Then the unbound primary antibody was removed by PBST wash (3 times) and incubated with HRP conjugated anti-rabbit secondary antibody (Biorad, 1706515), which was diluted 10000 times. Further, nonspecific binding was removed with PBST wash and the blots were developed (chemiluminescent) and analyzed using BioRad ECL kit. FIG. 4(c) shows the the dot blot of Aβ42 fibrils done using OC antibody in absence of TGR63 (L1) and presence of TGR63 (L2, L3), wherein L2 comprises 10 µM of TGR63 and L3 comprises 50 µM of TGR63. The blot image L1 displayed that only Aβ42 peptide sample contained maximum amount of fibrils (100%) while it decreased significantly (~50%) in L3 in presence of 50 µM TGR63. Quantification of Aβ42 fibrils was done by firstly incubating 10 µM of Aβ42 peptide samples for 48 h at 37° C. The incubated samples were then spotted on a PVDF membrane and probed with OC (fibril specific, 1:1000) antibody to measure the amount of Aβ fibrils. The results displayed in FIG. 4(d) reveal a decrease in Aβ42 aggregates from ~90% in L2 having 10 µM TGR63 to ~40% in L3 having 50 µM TGR63. Overall, the AFM, TEM and dot blot analysis was found to be in a good agreement with the ThT fluorescence assay and established that TGR63 as a potential candidate for modulating the multifaceted amyloid toxicities.

### Example 8

### Modulation of Membrane Toxicity

Inspiring from the above-mentioned studies, membrane toxicity modulation ability of TGR63 through immunocytochemistry in SHSY5Y cells was also investigated. The cells were cultured in 35 mm confocal dishes and treated individually with Aβ42 samples, pre-incubated for 24 h in the presence and absence of TGR63 for 2 h under cell growing conditions. Then the experimental cells were washed and fixed using 4% paraformaldehyde (PFA) to probe the Aβ42 fibrils with OC (1:250) antibody, followed by red fluorescent labeled (λₑₓ= 633 nm and λₑₘ= 650 nm) secondary antibody (Alexa 633-A21052, Thermo). Finally, unbound antibody was washed and 4′,6-diamidino-2-phenylindole (DAPI) was used to stain the nucleus before the confocal imaging. For DAPI staining, experimental cells were incubated with DAPI (500 nM) solution (PBS) for 10 min at room temperature. Then, the cells were washed with PBS three times and imaged under confocal fluorescence microscope (λₑₓ= 358 nm, λₑₓ= 461 nm). FIG. 5 depicts the images obtained by nuclear staining of SHSY5Y cells with DAPI(blue) and Aβ42 fibrils staining with OC antibody and red fluorescent labelled secondary antibody. The red fluoresence signals for only Aβ42 plaque without TGR63 were significantly high, indicating the attachment of Aβ42 fibrils on the plasma membrane. On the other hand, cells treated with TGR63 modulated Aβ42 fibrils showed lower red fluorescence signal on the plasma membrane as compared to the without TGR63 sample. This observation clearly demonstrated that TGR63 suppresses the amyloid toxicity by inhibiting the materialization of toxic plasma membrane adhesive Aβ42 fibrils, resulting in hampering the toxic irretrievable interactions between the plasma membrane and aggregated Aβ42 species.

### Example 9

### NMI Compounds as Therapeutic Agents in Alzheimer’s Disease

An Alzheimer’s disease-like environment was mimicked by exposing the cultured PC12 cells to 20 µM of Aβ42 fibrils, which formed cytotoxic aggregation species in the growth media. The results showed that Aβ42 caused mutilation to the cultured neuronal cells by decreasing the cell viability to 54% compared to untreated control cells (100%). The cells treated with Aβ42 in presence of TGR66-68 showed cell viability (~54%, 54% and 56%, respectively) similar to that of only Aβ treated cells. The cells treated with Aβ42 in presence of promising aggregation inhibitors TGR63-65 showed 80%, 76% and 62% cell viability, respectively. This corresponded to ~26%, 22% and 8% enhancement in the viability of cells from Aβ toxicity by TGR66-68, respectively, with TGR63 exhibiting superior neuronal rescue effects.

### Example 10

### TGR63 and TGR64 Modulating α-Syn Aggregation

Parkinson’s disease (PD) is a neuronal disorder, mainly affects the motor system. α-Syn is a presynaptic neuronal protein that is linked neuropathologically to PD plays a vital role in the pathogenesis in a number of ways. Self-aggregation of α-Syn to form fibrillar aggregates and these toxic species mediate disruption of cellular homeostasis and cause neuronal death. These toxic aggregates affect various intracellular targets, especially synaptic function. In this context, NMI compounds of the present disclosure were used to study the possible modulation of α-Syn aggregation. The effect of NMI compounds (TGR63 and TGR64) on α-syn (100 µM) aggregation inhibition was studied through ThT binding assay. Experiments were performed at stoichiometric ratios (α-Syn:inhibitor) of 1:1, 1:2, and 1:5 with the fixed concentration of α-Syn at 100 µM. The inhibition experiments demonstrated in FIG. 6 showed that TGR63 and TGR64 are capable of inhibiting α-Syn aggregation. TGR63 and TGR64 showed a concentration-dependent inhibition trend. At 1:1 ratio, inhibitors (TGR63 and TGR64) showed ~54 % decrease in the formation of α-Syn aggregates. Further, an increase in the concentration of inhibitors with 1:2 and 1:5 ratio effectively decreased α-Syn aggregation to 65% and 93% in TGR63 and 72% and 77% in TGR64. Therefore, TGR63 and TGR64 showed promising results in inhibiting α-Syn aggregation involved in Parkinson’s disease.

### Example 11

### In Vivo Study of TGR63

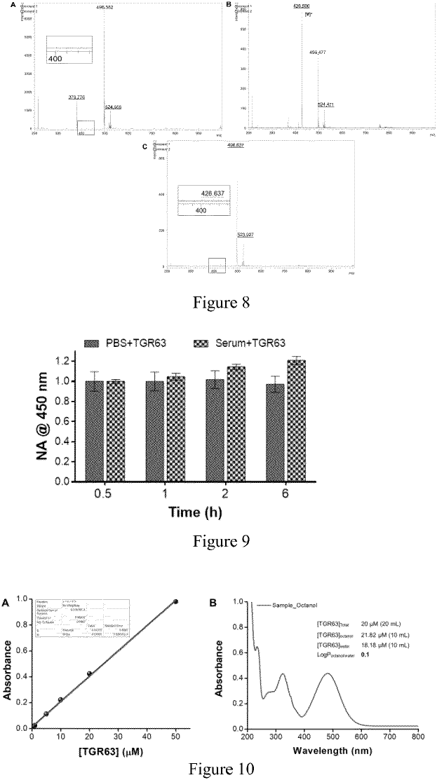

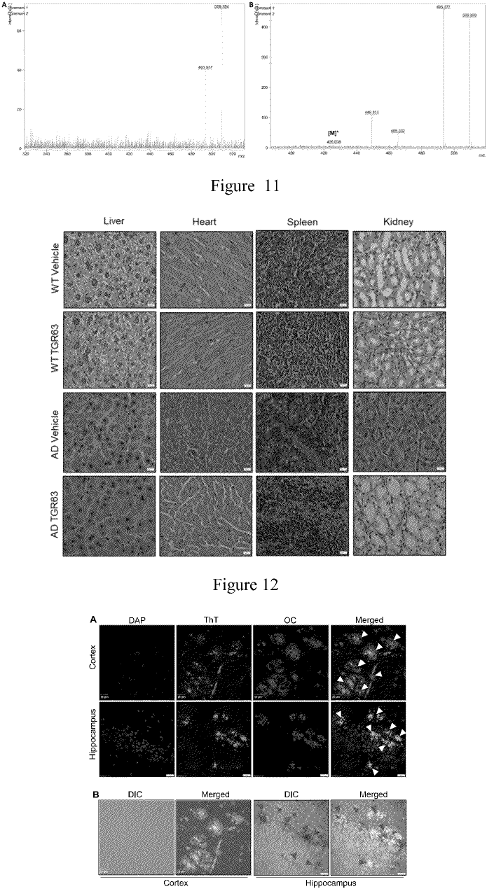

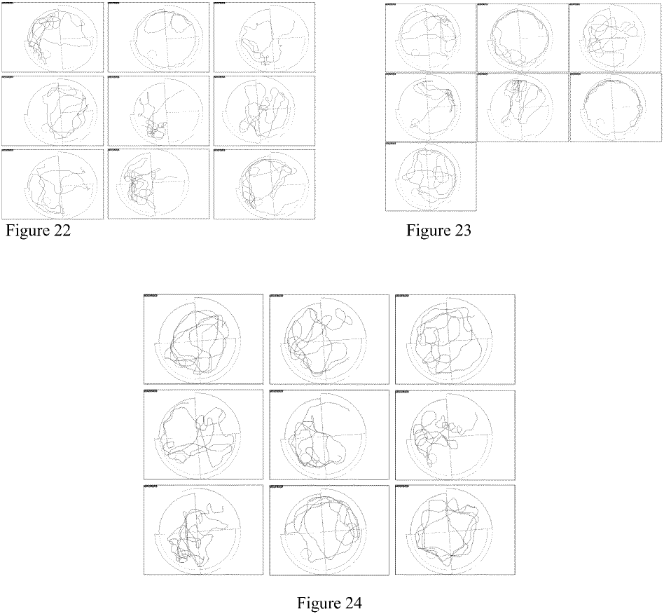

Pharmacokinetics of TGR63 in wild-type (WT) mice was performed to assess its in vivo efficacy. The lethal dose 50 (LD50) of TGR63 was determined in WT mice through intraperitoneal (IP) injection following the Organisation for Economic Cooperation and Development (OECD) guidelines. The survival of the experimental mice showed that TGR63 is mostly nontoxic in the experimental period due to the high LD50 value of ~157.9 mg/kg body weight (FIG. 7). FIG. 7A illustrates the calculation of lethal dose 50% (LD50) of TGR63 through intraperitoneal administration and is represented as table of experimental details and the final observation was done on 14th day. FIG. 7B illustrates the mortality (%) plotted against TGR63 concentration and calculation of LD50. Twenty-five WT mice were segregated in five different groups (G1-5, N= 5 per group) and administered with varying doses of TGR63 (1.7, 5.5, 17.5 56.0 and 179.0 mg/kg body weight, respectively) through IP injection and their survival was monitored for 14 days. The serum stability and blood-brain barrier (BBB) crossing ability of TGR63 were assessed through matrix-assisted laser desorption ionization (MALDI) mass spectrometry analysis of blood and brain samples of vehicle and TGR63 treated mice. TGR63 and vehicle were administrated in WT mice and sacrificed after 1 and 24 h to collect the blood for MALDI mass analysis FIG. 8 depicts the MALDI mass analysis of vehicle (FIG. 8A) and TGR63 treated mice blood serum after 1 h (FIG. 8B) and 24 h (FIG. 8C) of administration. The presence of TGR63 in blood was confirmed from the mass analysis even after 24 h. The MALDI analysis confirmed the presence of TGR63 in blood after 24 h of administration. TGR63 was incubated in PBS (10 mM, pH= 7.4) and blood serum in WT mice for different time intervals (0.5, 1, 2 and 6 h) at 37° C. to evaluate the serum stability under in vitro conditions. The spectrometric analysis (absorbance) confirmed the stability of TGR63 in blood serum. FIG. 9 depicts the Serum stability of TGR63 under in vitro conditions. TGR63 was incubated in PBS (10 mM, pH= 7.4) and blood serum (WT mouse) for different time (0.5, 1, 2 and 6 h) at 37° C. Data showed that the normalized absorbance (NA) of TGR63 at 450 nm recorded at different time intervals, which confirmed the stability of TGR63 in blood serum. The partition coefficient (P), a valuable physical property was calculated to predict the BBB permeability. The concentrations of TGR63 in octanol and water layer were found to be 21.82, and 18.18 µM, respectively and logP value was calculated to be 0.1. FIG. 10 depicts the calculation of LogP. (10A) Standard concentration curve obtained by measuring absorbance at 480 nm for 1, 5, 10, 20 and 50 µM of TGR63 in octanol and (10B) Absorbance of octanol layer (Sample_Octanol) and calculation of LogP. The calculated positive logP value predicts the possible BBB crossing ability for TGR63. For in vivo assessment, TGR63 and vehicle administrated WT mice were sacrificed after 1 h to collect the brains for MALDI mass analysis. TGR63 treated mouse brain sample showed a mass peak at 426.04 (m/z), which was absent in the vehicle-treated sample and confirmed BBB crossing ability of TGR63 FIG. 11 depicts the MALDI mass analysis of vehicle (11A) and TGR63 (11B) treated mouse brain lysate after 1 h. The absence of any characteristic mass peaks in vehicle treated control sample confirm the presence of TGR63 in treated mice brain.

Further, TGR63 (5 mg/kg body weight) and vehicle (control) were administrated in age (6 months old) matched APP/PS1 and WT mice daily for 8 months to examine the organ toxicity upon prolonged TGR63 administration. The experimental mice were sacrificed at 14 months of age and critical organs viz., liver, heart, spleen and kidney were harvested to perform gold standard hematoxylin and eosin (H&E) staining (stain nucleus and cytoplasm, respectively). The H&E staining of TGR63 treated mice (WT and AD) tissue samples exhibited nucleus and cytoplasm staining similar to healthy tissue (vehicle-treated WT mice). The healthy or TGR63 treated tissue samples did not show any abnormal scar, disorganization, inflammatory infiltrate, hepatotoxicity or necrosis (FIG. 12), which confirmed the tremendous in vivo biocompatibility and nontoxic nature of TGR63. FIG. 12 (Scale bar: 10 µm) depicts the evaluation of organ toxicity of TGR63. Bright field images of vehicle and TGR63 treated mice organs (liver, heart, spleen and kidney) stained with hematoxylin and eosin. TGR63 treated mouse organs showed the healthy nature like vehicle treated control and confirmed the biocompatibility and nontoxic nature of TGR63. The pharmacokinetics study of TGR63 revealed serum stability, BBB permeability and biocompatibility, underscoring its suitability for the long-term treatment in APP/PS1 AD phenotypic mice. These studies enabled to evaluate the efficacy of the lead candidate to ameliorate the cognitive impairment, for which APP/PS1 AD and WT mice were administrated (IP) with TGR63 (daily dose of 5 mg/kg body weight) starting from the age of 6 months to 14 months.

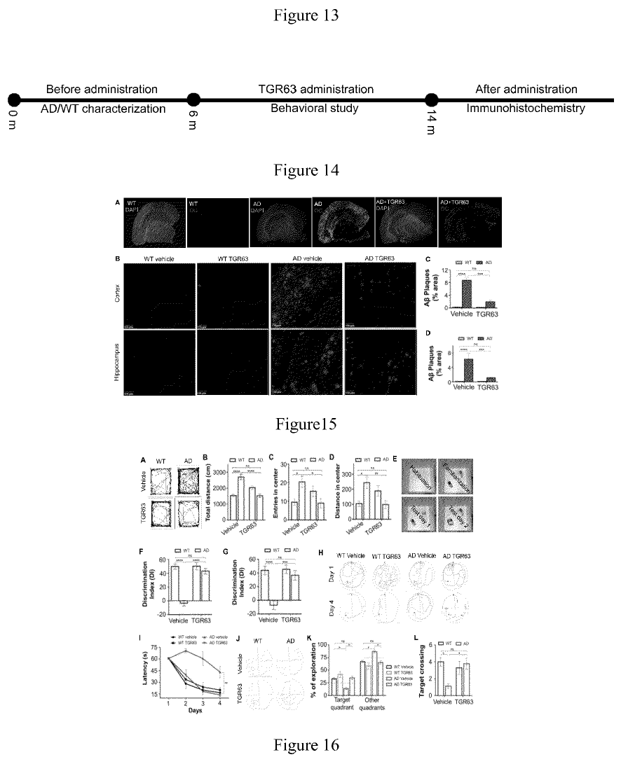

To evaluate the activity of TGR63 to ameliorate amyloid burden in in vivo AD model, APP/PS1 mice were bred, maintained and characterized (WT: wild type; AD: APP/PS1 positive) according to the Jackson Laboratory protocols. The double transgenic APP/PS1 mice (B6C3-Tg (APPswe, PSEN IdE9)85Dbo/J; stock number 004462), which express human transgenes APP and presenilin 1 (PS1) in the central nervous system (CNS) contains the Swedish and L166P mutations, respectively. The K595N/M596L (Swedish) mutation favors the amyloidogenic processing of APP protein, and PS1 mutation (L166P) elevates the production of Aβ peptides through modifying the intra-membrane γ-complex. The presence of Aβ plaques in the APP/PS 1 AD phenotypic mouse brain was confirmed and compared with the healthy brain by Aβ plaques-specific staining protocols. FIG. 13 depicts the Staining of amyloid plaques with OC primary antibody and ThT or CQ probe, FIG. 13A represent the high-resolution confocal microscopy images of cortex and hippocampus regions of the AD mouse brain, immunostained with OC antibody (red), DAPI (blue) and ThT (green). The merged images display significant overlap between ThT and OC staining to confirm the amyloid deposition (pointed with white arrows), FIG. 13A represent the visualization of amyloid deposits associated neuronal damage: The DIC images of different regions of AD. The merged images of DIC and confocal microscopy images show amyloid plaques associated brain damage (pointed out with red arrows). The brains were harvested from the age matched WT and AD mice and treated with PFA (4%) and sucrose solution (30%) for the sagittal brain sectioning (40 µm sections). The brain sections were co-stained with ThT (λₑₓ= 442 nm, λem= 482 nm) and OC primary antibody followed by fluorescently labeled secondary antibody (λₑₓ= 633 nm, λem= 650 nm) or CQ to visualize and confirm the amyloid plaques deposition. The confocal images acquired from different regions of the brain (cortex and hippocampus) showed localized bright green and red fluorescence signals confirming the deposits of amyloid plaques in the APP/PS1 mice brain. Similar fluorescence signals (green and red) were absent in the age-matched WT brain section, confirming the amyloid plaques-free healthy brain (FIG. 13A). The hippocampal damage, a hallmark of advanced AD condition was partially observed in 14 month old APP/PS1 mice. Age-matched AD, and WT cohorts were administered with TGR63 (5 mg/kg body weight/day) and vehicle starting from the age of 6 months following the treatment protocols as shown in FIG. 14. The experimental mice were sacrificed after completing the behavioral studies (14 months) to investigate amyloid deposits in the brain using immunohistochemistry. The sagittal brain sections were permeabilized and blocked with PBTx (0.1 M PBS and 0.1% TritonX-100) and goat serum (1%) containing BSA (2%) at room temperature, respectively. The processed sections were incubated with amyloid fibrils specific primary antibody (OC, 1:250) at 4° C. for 48 h to stain the dense core of amyloid plaques. The processed brain sections were further treated with red fluorescent-labeled (λₑₓ= 633 nm and λem= 650 nm) secondary antibody (1:1000) and DAPI to perform confocal imaging (FIG. 15A). The confocal images of WT cohort brain tissue sections did not show any deposits of Aβ plaques in both cortex and hippocampus regions. The age-matched AD cohort brain tissue sections prominently displayed deposits of Aβ plaques in different parts of the brain viz., neocortex, striatum, primary sensory-motor areas, hippocampus, temporobasal and frontomedial areas. These results provided strong evidence of chronic accumulation of Aβ plaques in the brain associated with AD progression. Predictably, the vehicle-treated AD brain tissue images (N= 3) showed an accumulation of Aβ plaques 8.87% and 6.28% area of the cortex and hippocampus, respectively (FIG. 15B). Remarkably, TGR63 treatment (N= 3) significantly reduced the Aβ plaques deposits to 1.94% and 0.94% area of the cortex and hippocampus, respectively (FIGS. 15C and 15D). In other words, TGR63 treatment reduced Aβ deposits by 78% and 85% in the cortex and hippocampus, respectively. The immunostaining of Aβ deposits in TGR63 treated AD brain tissue displayed a considerable reduction in the amyloid load and encouraged us to test for the corresponding improvement of memory and cognitive functions.

### Recovery of Cognitive Functions

AD is characterized by the progressive deterioration in cognitive functions, which generally include learning and memory impairment leading to neuropsychiatric symptoms viz., aggression, agitation, anxiety and depression. APP/PS1 mice show age-related AD-like phenotypes linked to Aβ plaques deposition in the brain. Hence the recovery of cognitive functions in TGR63 treated APP/PS1 mice (FIG. 16) was assessed. Open-field (OF) test was performed to evaluate the effect of TGR63 on anxiety and locomotion. Next, the amelioration of learning disability and memory impairment by TGR63 treatment was evaluated through novel object identification (NOI) and Morris water maze (MWM) behavioral tests.

In OF test, all the experimental mice were individually allowed to explore a novel platform (45 X 45 cm) and their locomotion activity was monitored for 5 min (Sony HDRCX405 camera) and analyzed using the smart 3 software (Panlab; FIG. 16A). The trajectories of vehicle-treated AD mice (AD vehicle) showed higher activity (travel average 2698.25 cm) compared to vehicle-treated WT mice (travel average 1533.88 cm), which indicated the AD-like phenotype of APP/PS1 mouse model (FIG. 16B). Interestingly, TGR63 treated AD (AD TGR63) mice showed significantly shorter travel paths (average 1515.33 cm) compared to AD vehicle cohort suggesting improved locomotor functions and anxiety similar to vehicle-treated WT mice (WT vehicle). The anxiety behaviors of TGR63 treated mice were assessed by the time spent and the entries in the center zone (20 X 20 cm) of OF arena. As expected, AD vehicle showed the maximum number of entries (~20) and travel path (average 243.0 cm) among other cohorts in the center zone, which confirmed the characteristic anxious nature of AD conditions (FIGS. 16C and 16D). Remarkably, TGR63 treated AD mice showed behaviors similar to healthy WT vehicle cohorts with ~9 entries and travel average of 98.14 cm exploration in the center zone. The OF test data revealed that TGR63 ameliorated the β-amyloid-induced aggression, agitation and anxiety observed in the middle stages of AD. Next, the effect of TGR63 on memory processing, viz., acquisition, consolidation and retrieval were evaluated through NOI test, which has been widely used as a tool to study the neurobiology of memory using the natural tendency of rodents to explore novel objects more than the familiar objects. All the experimental mice were familiarized with two identical objects (familiar objects) in a known habituated arena and allowed to explore a novel and familiar object after 24 and 48 h of familiarization (FIG. 16E). The exploration time with each object was measured using a stopwatch, and the discrimination index (DI) was determined using the formula, (time exploring the novel object - time exploring the familiar) / (time exploring novel + familiar) * 100. The test result after 24 h showed significantly lower DI (-3) for AD vehicle cohort compared to WT vehicle cohort (+49), which affirmed the deteriorating memory formation and recollection under progressive AD conditions (FIG. 16F). On the other hand, calculated DI of WT TGR63 cohort (+50) is similar to the WT vehicle cohort confirming TGR63 did not affect memory formation. Remarkably, AD TGR63 cohort exhibited an improved DI (+43) compared to AD vehicle cohort (-3) confirming the therapeutic efficacy of TGR63 in memory processing (acquisition, consolidation and retrieval) under AD condition. Similarly, the calculated DI after 48 h was lowest (-7) for AD vehicle cohort compared to both vehicle and TGR63 treated WT cohorts (+43 and +45, respectively) (FIG. 16G). AD TGR63 cohort showed DI of +38, which indicate normal memory formation and retrieval. The DI of TGR63 treated WT, and AD cohorts at 48 h have marginally reduced (~5 units of DI) compare to 24 h, reveal the natural long-term depression of healthy animals. The blocking of essential synaptic receptors (NMDA and AMPA) by Aβ aggregation species leading to synaptic dysfunction followed by impairment in hippocampal LTP formation. The NOI test result demonstrated that AD positive mice (APP/PS 1) exhibit the memory impaired phenotypes compare to WT mice. TGR63 treatment ameliorates the memory impairment in APP/PS 1 mice by reducing the toxic amyloid burden from the brain under progressive AD conditions.

The spatial and episodic memory formation under AD conditions were investigated through spatial learning and memory development tasks in MWM test. MWM test was performed in a water pool (radius: 70 cm) and experimental mice were trained four times in a day to find a hidden platform, which was removed in probe trial to assess the spatial memory. The latency time to reach the hidden platform during the training period was recorded to determine spatial learning (FIG. 16H). As anticipated, AD vehicle cohort required more time (~70, 60 and 43 s) to reach the platform during training days (2nd, 3rd and 4th, respectively), while other cohorts showed a smooth spatial memory formation with time (FIG. 16I). AD TGR63 cohort behaved like a healthy WT mouse and exhibited significant improvement in spatial memory formation compared to AD vehicle cohort. In the probe trial, AD vehicle cohort spent most of the time (~87% of total time) in other quadrants (without platform), while other cohorts (WT vehicle, WT TGR63 and AD TGR63) spent only ~67%, 58% and 66% of total time in without platform quadrants, respectively. The AD vehicle cohort spent minimum time (~13% of total time) in target quadrant (with platform) compared to WT vehicle cohort (~33% of total time). TGR63 does not affect the spatial memory formation and retrieval in the healthy brain, as the WT TGR63 cohort showed similar exploration (<35% of total time) tendency like WT vehicle cohort (FIG. 16J). Interestingly, AD TGR63 cohort explored <20% (~34% of total time) in the target quadrant than AD vehicle cohort, which is similar to that of healthy mice (FIG. 16K). Further, the spatio-temporal memory was determined by analyzing their activity in the platform region, which revealed AD vehicle cohort crossed the platform for minimum times (~1 time) compared to the WT vehicle cohort (~4 times) (FIG. 16L). Remarkably, AD TGR63 cohort crossed the platform region ~4 times, which is greater than the AD vehicle cohort. MWM study demonstrated significant effect of TGR63 treatment on the medial entorhinal cortex and hippocampus in the AD brain, the key areas for the development of spatial learning and memory.

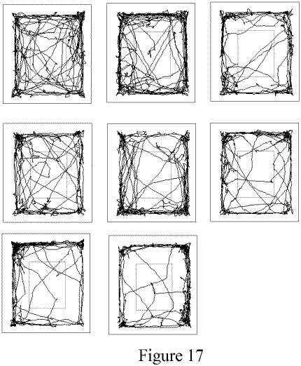

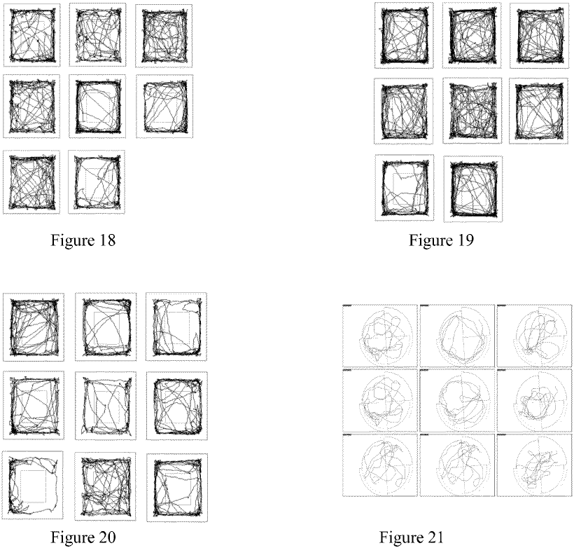

FIGS. 17 and 18 depict the locomotion of vehicle treated and TGR63 treated WT mice cohort during OF test respectively. FIGS. 19 and 20 depicts the locomotion of vehicle treated and TGR63 treated AD mice cohort during OF test respectively. FIGS. 21 and 22 depicts trajectory of vehicle treated and TGR63 treated cohort during MWM probe trail, respectively. FIGS. 23 and 24 depicts trajectories of vehicle treated and TGR63 treated AD mice cohort during MWM probe trail, respectively.

AD is characterized by the progressive deterioration in cognitive functions, which generally include learning and memory impairment leading to neuropsychiatric symptoms viz., aggression, agitation, anxiety and depression. APP/PS1 mice show age-related AD-like phenotypes linked to Aβ plaques deposition in the brain. Open-field (OF) test was performed to evaluate the effect of TGR63 on anxiety and locomotion. Next, the amelioration of learning disability and memory impairment by TGR63 treatment was evaluated through novel object identification (NOI) and Morris water maze (MWM) behavioral tests.

From the spatial learning and memory development tasks it can be understood that the compounds of the Formula (I) treated mice exhibited improved cognitive performance. This is due to the modulation of pathogenic proteins such as Aβ42, tau and/or α-syn aggregates by the compounds of the Formula (I). Thus, the significant enhancement of memory and cognitive performance in the behavioral studies is in excellent agreement with the amelioration of amyloid burden, associated neuronal toxicity and improved cognitive functions in the progressive AD conditions validated the anti-AD (reversal of cognitive decline) credentials of TGR63 (FIGS. 17 to 24).

### Inhibition of Tau Aggregation

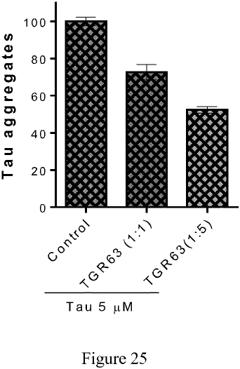

Tau is a microtubule-associated proteins that interacts with tubulin and stimulates its assembly into microtubules. The extent of phosphorylation regulates the activity of tau protein and the hyperphosphorylation diminishes its physiological function leading to several neuronal disorders like AD and tauopathies. The self-aggregation of hyperphosphorylated tau form intracellular neurofibrillary tangles (NFTs) and paired helical filaments (PHFs) that impairs the axonal functions and degenerates neuronal cells. Considering the prominent role of tau in AD pathology, the self-aggregation and deposition of hyperphosphorylated tau became a potential target to tackle AD and related tauopathies. Effect of TGR63 on tau aggregation was assessed. Full length tau (5 µM) was incubated with TGR63 (5 and 25 µM) for 72 h at 37° C. in the presence of aggregation inducer (heparin; 1.25 µM). The extent of aggregation of incubated samples were assessed by ThT fluorescence (λₑₓ= 442 nm and λₑₘ=482 nm) assay. FIG. 25 depicts the inhibition of tau (5 µM) aggregation in presence of TGR63. As expected, the ThT fluorescence signal was increased by ~15 folds with compare to only ThT, which confirmed the formation of pathogenic tau aggregates. Interestingly, the fluorescence signal of TGR63 (5 and 25 µM) treated samples decreased to ~72 and 52%, respectively compare with untreated controls (100%). In other word, 1: 1 and 1: 5 stoichiometric ratio of tau and TGR63 effectively reduced the tau aggregates by ~28 and 48%, respectively. This result demonstrated that TGR63 is a potential candidate to modulate the pathogenic tau aggregation and associated toxicity in AD pathology.

### Advantages of the Present Disclosure

The above-mentioned implementation examples as described on this subject matter and its equivalent thereof have many advantages, including those which are described.

The analysis of the small molecule-based naphthalene monoimide (NMI) compounds of Formula (I) of the present disclosure achieved through advanced design strategy and structure optimization demonstrates excellent results in inhibition of Aβ42 fibrillar assembly involved in amyloidogenesis and rescue of neuronal cells from amyloid toxicity. The derivatives possess improved hydrophobicity and exhibit enhanced capability to penetrate the plasma membranes of live cells. The compounds of Formula (I) of the present disclosure modulates aggregation of Aβ42, tau, or α-syn and helps in treating condition or disorder or diseases mediated by aggregation of Aβ42, tau, or α-syn. The compounds of present disclosure also provides reversal or improvement of cognitive decline. This proves that including NMI compounds in the manufacturing of pharmaceutical composition, can be used for effective treatment of conditions mediated by neurodegenerative diseases such as Alzheimer’s disease, Parkinson’s diseases, and others. Moreover, the designed derivatives were obtained through cost-effective modification of naturally available products and are completely non-toxic. Thus, a pharmaceutical composition comprising the NMI compounds of Formula (I) of the present disclosure, along with other clinically relevant modulators and pharmaceutical carriers can be administered in effective amounts to treat both moderate and advanced stages of neurodegenerative diseases.

Although the subject matter has been described in considerable detail with reference to certain embodiments thereof, other embodiments are possible. As such, the spirit and scope of the disclosure should not be limited to the description of the embodiments contained herein.

We claim:

1-16. (canceled)

17. A compound and its polymorphs, stereoisomers, prodrugs, solvates, co-crystals, intermediates, pharmaceutically acceptable salts, and metabolites thereof, selected from:
a. 2-(6-((4-(dimethylamino)phenyl)ethynyl)-1,3-dioxo-1H-benzo[de]isoquinolin-2(3H)-yl)-N,N,N-trimethylethan-1-aminium;
b. 2-(2-aminoethyl)-6-((4-(dimethylamino)phenyl)ethynyl)-1H-benzo[de]isoquinoline-1,3(2H)-dione;
c. 6-((4-(dimethylamino)phenyl)ethynyl)-2-(2-(2-hydroxyethoxy)ethyl)-1H-benzo[de]isoquinoline-1,3(2H)-dione;
d. 2-(1,3-dioxo-1H-benzo[de]isoquinolin-2(3H)-yl)-N,N,N-trimethylethan-1-aminium;
e. 2-(6-(dimethylamino)-1,3-dioxo-1H-benzo[de]isoquinolin-2(3H)-yl)-N,N,N-trimethylethan-1-aminium; and
f. 2-(1,3-dioxo-6-(phenylethynyl)-1H-benzo[de]isoquinolin-2(3H)-yl)-N,N,N-trimethylethan-1-aminium.

18. A process of preparation of compound as claimed in claim 17, and its polymorphs, stereoisomers, prodrugs, solvates, co-crystals, intermediates, pharmaceutically acceptable salts, and metabolites thereof, the process comprising: 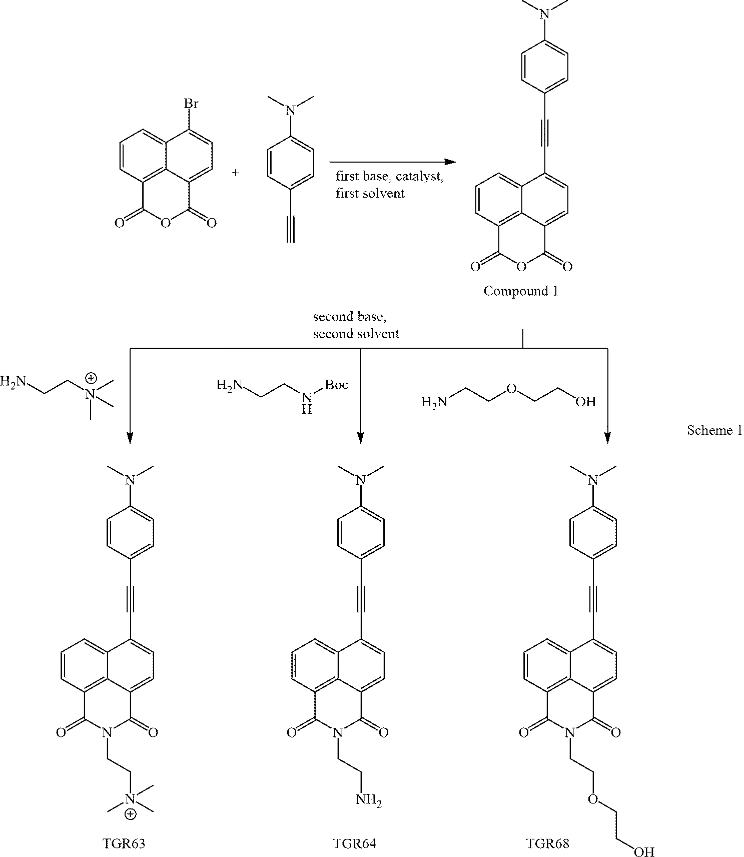 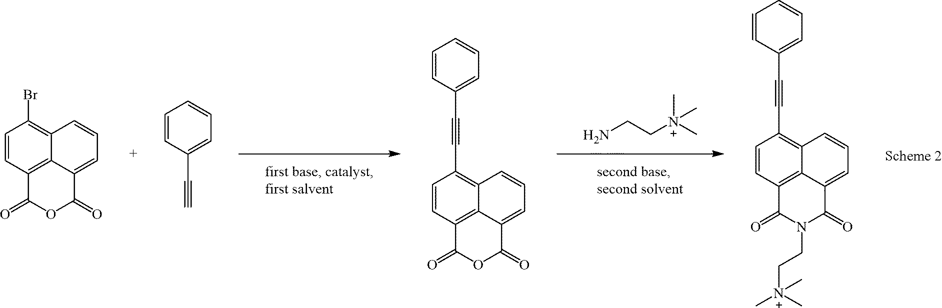 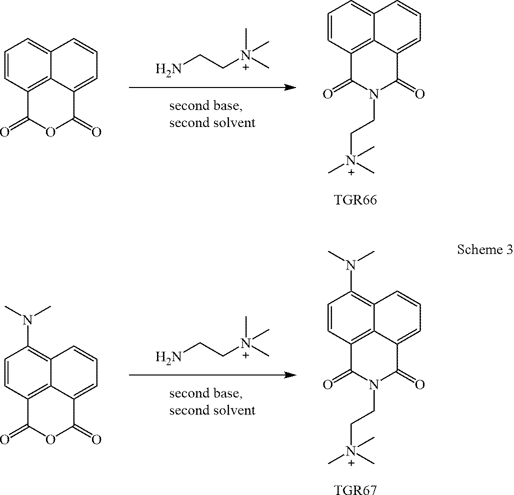
.

19. The process as claimed in claim 18, wherein the first base is selected from N,N-diisopropylethylamine (DIPEA), triethylamine (Et₃N), C₁₋₁₀ alkyl amine, or combinations thereof; the first solvent is selected from dimethyl formamide, isopropyl alcohol, or combinations thereof; and the catalyst is selected from copper iodide, copper sulphate, sodium ascorbate, or combinations thereof.

20. The process as claimed in claim 18, wherein the second base is selected from N,N-diisopropylethylamine (DIPEA), triethylamine (Et₃N), C₁₋₁₀ alkyl amine, or combinations thereof; and the second solvent is selected from dimethyl formamide, isopropyl alcohol, or combinations thereof.

21. The compound as claimed in claim 17 or a pharmaceutically acceptable salt thereof for use in the manufacture of a medicament for treating a neurodegenerative disease.

22. The compound as claimed in claim 17, wherein the neurodegenerative disease is selected from Alzheimer’s disease (AD), Parkinson’s disease (PD), prion diseases, polyglutamine expansion diseases, Huntington’s disease (HD), tauopathies, frontotemporal dementia associated with tau-immunoreactive inclusions (FTD-tau), progressive supranuclear palsy (PSP), corticobasal degeneration (CBD) or amyotrophic lateral sclerosis (ALS).

23. The compound as claimed in claim 17, wherein the compounds of Formula (I) modulate aggregation of Aβ42, tau, and α-syn.

24. The compound as claimed in claim 17, wherein the compounds of Formula (I) provides reversal of cognitive decline or improvement of cognitive decline.

25. A pharmaceutical composition comprising the compound as claimed in claim 17 or a pharmaceutically acceptable salt thereof with a pharmaceutically acceptable carrier, optionally in combination with one or more other pharmaceutical compositions.

26. The pharmaceutical composition as claimed in claim 25, wherein the composition is in a form selected from tablet, capsule, powder, syrup, solution, aerosol, or suspension.

27. A method for the treatment of a condition mediated by a neurodegenerative disease or by aggregation of Aβ42, tau, or α-syn, said method comprising administering to a subject an effective amount of one or more of the compound as claimed in claim 17 optionally with other clinically relevant immune modulator agents or biological agents to a subject in need of thereof.

28. The method as claimed in claim 27, wherein the neurodegenerative disease is selected from Alzheimer’s disease (AD), Parkinson’s disease (PD), prion diseases, polyglutamine expansion diseases, Huntington’s disease (HD), tauopathies, frontotemporal dementia associated with tau-immunoreactive inclusions (FTD-tau), progressive supranuclear palsy (PSP), corticobasal degeneration (CBD), or amyotrophic lateral sclerosis (ALS).
